(19) United States
(12) Patent Application Publication
(10) Pub. No.: US 2021/0407672 A1
(43) Pub. Date: Dec. 30, 2021

(54) CLINICAL ASSESSMENT TOOL

(71) Applicant: Clover Health, Jersey City, NJ (US)

(72) Inventors: Paul Zumbrun, San Francisco, CA (US); Mark Spektor, New York, NY (US); Kejia Zhu, San Francisco, CA (US); Christopher James Lauinger, Golden, CO (US); Nathaniel Quan, San Francisco, CA (US); Clara Wu, San Francisco, CA (US); Daniel Lee, San Francisco, CA (US); Andrew Toy, San Francisco, CA (US)

(21) Appl. No.: 16/912,456

(22) Filed: Jun. 25, 2020

(51) Int. Cl. G16H 50/20; G16H 10/60; G16H 15/00; G16H 20/10; G16H 40/20; G16H 10/40; G16H 50/70; G16H 70/20

(52) U.S. Cl. CPC G16H 50/20; G16H 10/60; G16H 15/00; G16H 70/20; G16H 40/20; G16H 10/40; G16H 50/70; G16H 20/10

(57) ABSTRACT

Techniques are described herein that provide relevant information associated with a patient to a medical provider. In some instances, the relevant information may be provided to the medical provider during a clinical visit with the patient, such as via an application managed by a service provider. In some instances, the relevant information may be provided to the medical provider at another time, such as that associated with a referral submission. The relevant information may be provided via one or more interfaces associated with an application. In some instances, the interface(s) may guide the medical provider through a clinical visit to maximize a level of care provided to the member and minimize an amount of time associated with the clinical visit.

### BACKGROUND

People generally visit medical providers for routine check-ups and procedures, and also for specific issues, such as illness, surgical follow-up, and the like. During a clinical visit with a patient, a medical provider may have limited access to information about the patient. For example, the information available to the medical provider may be limited to data available in an electronic medical record managed by the medical provider and/or office or hospital associated therewith, such as based on previous visits to the medical provider. Additionally, the medical provider may be limited in an amount of time available during the clinical visit to discuss medical issues with the patient. A combination of a lack of time and lack of information available to the medical provider may result in an inability for the medical provider to review important information with the patient. In some instances, without access to relevant information about a patient and without time to determine the information, the medical provider may be unable to effectively treat the patient during a clinical visit.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 is a schematic view of an example system usable to implement a clinical assessment tool, as described herein.

FIGS. 2A and 2B illustrate an example interface in which clinical assessments may be reviewed and generated. FIG. 2A illustrates an example interface in which previously generated clinical assessments may be reviewed. FIG. 2B illustrates the example interface configured for generating a new clinical assessment.

FIG. 3 illustrates an example interface associated with a clinical assessment main page.

FIGS. 4A and 4B illustrate an example interface associated with a clinical assessment diagnosis page. FIG. 4A illustrates an example in which a medical provider may confirm a diagnosis. FIG. 4B illustrates an example interface in which a medical provider may be unable to confirm a diagnosis.

FIG. 5 illustrates an example interface associated with a clinical assessment medications page.

FIGS. 6A-6C illustrate an example interface associated with a clinical assessment gaps in care page. FIG. 6A illustrates the example interface in which a medical provider may indicate that the patient refuses a suggested procedure. FIG. 6B illustrates the example interface in which the medical provider indicates that a referral for the suggested procedure will be submitted. FIG. 6C illustrates an example interface for submitting the referral.

FIG. 7 illustrates another example interface associated with submitting a referral for treatment.

FIG. 8 illustrates an example interface associated with a clinical assessment clinical recommendations page.

FIG. 9 illustrates an example interface associated with a clinical assessment submission and medical record verification.

FIG. 10 illustrates a block diagram illustrating an example system of computing devices usable to implement example techniques described herein.

FIG. 11 illustrates an example process for surfacing a clinical assessment tool and updating a member record based on input received via the clinical assessment tool, utilizing the techniques described herein.

FIG. 12 illustrates an example process for processing a referral submitted by a medical provider, utilizing the techniques described herein.

FIG. 13 illustrates an example process for processing a referral based at least in part on input from a member, utilizing the techniques described herein.

FIG. 14 illustrates an example process for determining whether to automatically approve a referral.

FIG. 15 illustrates an example process for training a machine learning model to determine a potential diagnosis for a member.

FIG. 16 illustrates an example process for training a machine learning model to determine whether a medical procedure may be automatically approved.

### DETAILED DESCRIPTION

This application describes techniques for providing relevant information associated with a patient to a medical provider. In some instances, the relevant information may be provided to the medical provider during a clinical visit with the patient, such as via an application managed by a service provider. The application may enable expedited and more effective interactions between the medical provider and the patient during the clinical visit.

A medical provider or associate thereof (e.g., office staff, assistant, associate, etc.) may determine that a patient (e.g., member) associated with a service provider is scheduled for an appointment (e.g., clinical visit). The medical provider or associate may access an instance of the application to generate a clinical assessment (e.g., an interface associated with a clinical visit, visit form, etc.). The medical provider or associate may submit, to a service provider and via the instance of the application, identifying information associated with the member (e.g., name, identifier, date of birth, etc.) and/or information associated with the appointment (e.g., provider, location, date, time, etc.). The service provider may configured to provide one or more services to the member and/or the medical provider, such as insurance services, clinical visit assistance services, referral services, scheduling services, and the like. The service provider may receive the information and generate the interface to assist a medical provider during the clinical visit.

In various example, the service provider may generate the interface based on member data, such as that stored in a member record associated with the member. The member may include a member associated with the service provider. The member data may include demographic information, medical history (e.g., previous diagnoses, medical procedures, surgeries, etc.), laboratory results (e.g., glucose, cholesterol, etc.), medical test results (e.g., Echo stress test result, EKG, etc.), member location information (e.g., home address, work address, etc.), pharmacological information (e.g., prescriptions, prescription fill information (e.g., last fill, expirations, etc.), preferred pharmacy, etc.). In some examples, the service provider may access the member data to determine information to include in the interface associated with the clinical visit between the medical provider and the member. In such examples, the interface may be tailored to an individual member at a particular time.

In various examples, the interface may include one or more potential diagnoses for the member (e.g., undiagnosed conditions, unconfirmed diagnoses). In some examples, the interface may include evidence to support the one or more potential diagnoses. In such examples, the medical provider may access specific reasoning for a potential diagnosis, such as to discuss the symptoms, evidence, and/or health trends with the member. In some examples, the service provider may determine the one or more potential diagnoses for the member based on the member data. For example, the service provider may determine, based on a medical history and series of lab results over a period of time, that the member may have type 2 diabetes. The service provider may include the potential diagnosis and lab results indicating glucose levels in the interface to assist the medical provider in the clinical visit.

In various examples, the interface may include a request for the medical provider to confirm a potential diagnosis. In such examples, the interface may include a selectable option for the medical provider to quickly and easily confirm the diagnosis with the member. Responsive to selection of the selectable option to confirm, the service provider may include the diagnosis confirmation in the member data.

In some examples, the interface may include a selectable option indicating that the medical provider was not able to confirm a potential diagnosis. In such examples, responsive to selection of the selectable option indicating an inability to confirm, the service provider may surface a request for input regarding the inability to confirm via the interface. In some examples, the request for input may include a list of reasons why the medical professional is unable to confirm (e.g., additional results needed, condition resolved, assessed and not diagnosed, etc.). In such examples, the medical professional may select a reason from the list to provide additional information to the service provider. In some examples, the interface may include a means by which the medical provider may document particular information regarding the inability to confirm the potential diagnosis. Continuing the example from above, the medical provider may determine that a most recent glucose test was conducted too long in the past to effectively diagnose type 2 diabetes at the clinical visit. The medical provider may indicate via the interface that additional laboratory results would be needed and may specify that results to a recent (within a threshold time) fasting glucose test would be necessary to confirm the diagnosis.

In various examples, the service provider may receive the input from the medical provider and may update the member data and/or process an insurance approval for additional procedures and/or testing based on the input. For example, the service provider may receive the input from the medical provider about an updated glucose test and may process an insurance approval for the glucose test.

In various examples, the interface may include medication information associated with the member. In some examples, the service provider may determine the medication information based on the member data. In various examples, the medication information may include some or all of the current prescriptions and/or past or expired prescriptions associated with the member. In some examples, the medication information may include a prescriber associated with the prescriptions and/or relevant dates (e.g., fill date, expiration date, etc.).

In some examples, the medication information may include one or more notifications regarding the prescriptions. The notifications may include eligibility for an extended prescription (e.g., from a 30-day prescription to a 100-day prescription, etc.), generic drug availability, member delays in filling prescription, overdue prescriptions, and the like. In some examples, the interface may include a means by which the medical provider may quickly and easily address a notification. In such examples, the service provider may cause a selectable option for the medical provider to select to renew and/or modify a prescription, order a new prescription, or the like. For example, the medication information may include an eligibility to extend a prescription from a 60-day prescription to a 120-day prescription. The interface may include a selectable option for the medical provider to indicate that they will or will not be modifying the prescription. In some examples, the interface may additionally surface a request for input from the medical provider, such as to justify a reason to modify or to not modify the prescription. In various examples, responsive to receiving the input, the service provider may update the member data, and/or process approval for the modified prescription.

In various examples, the interface may include information associated gaps in the member medical care (e.g., gaps in care). The gaps in care may include one or more screenings, procedures, tests, surgeries, consults, or the like that the member should undergo. In some examples, service provider may determine the gaps in care based on recommended care guidelines (e.g., health maintenance guidelines, recommended health screenings, etc.), such as those published by the center for disease control or other health organization. In some examples, the service provider may determine the gaps in care based on the member data.

In various examples, the interface may provide a means by which the medical provider may submit a referral based on a gap in care. The interface may include information about locations and/or providers eligible to provide a service associated with the gap in care (e.g., a screening, a procedure, a test, a surgery, a consult, etc.). The service provider may receive an indication that the medical provider submitted the referral via the interface and process the referral. In some examples, processing the referral may include processing an approval for insurance payment, scheduling an appointment, providing an indication to the medical provider and/or office associated with the referral to schedule the appointment, sending referral and/or approval information to the member, and the like. In some examples, the service provider may update member data based on the referral information.

In various examples, the interface may include one or more clinical recommendations based on member data. The clinical recommendations may include information to inform medical decisions, such as information associated with a member diagnosis (e.g., known diagnosis), a potential treatment for the member, studies published regarding a diagnosis or medical condition associated with the member, and the like. In various examples, the interface may include a justification for the clinical recommendations, such as a justification for treatment. The justification may include member data supporting the clinical recommendation (e.g., a diagnosis, medications, and/or lab results). In some examples, the justification may include clinical guidelines for treating a particular diagnosis or condition.

In some examples, the interface may include a request for input regarding the clinical recommendations, such as whether or not the medical provider will act on a clinical recommendation. For example, a service provider may determine to recommend that a member with type 2 diabetes and an elevated glucose level be treated with an injectable therapy. The interface may include a request for input as to whether the injectable therapy was prescribed or not.

In various examples, the service provider may identify multiple potential diagnoses, medications, gaps in care, and/or clinical recommendations and may determine a number of each to surface via the interface. In some examples, the number may be the same or different for each of the potential diagnoses, medications, gaps in care, and clinical recommendations. In some examples, the number for each of the potential diagnoses, medications, gaps in care, and clinical recommendations may be based on a time associated therewith and/or a total time for the clinical visit. For example, the service provider may determine that a total time associated with a clinical visit is 5 minutes, and may determine to surface two potential diagnoses estimated to be confirmed in 3 minutes, one medication estimated to be updated in 30 seconds, one gap in care estimated to be updated in 30 seconds, and a clinical recommendation estimated to be updated in 1 minute. Though this is merely for illustrative purposes and any other amount of time and combination of information is contemplated here. In some examples, the medical provider may provide the total time for the clinical visit to the service provider. In such examples, the service provider may store the total time for clinical visits in a datastore (e.g., in medical provider data). For example, a first medical provider may inform the service provider that clinical visits should be a total of 6 minutes and a second medical provider may inform the service provider that clinical visits should be a total of 8 minutes. In such examples, the service provider may update first medical provider data and second medical provider data based on the respective total times and may determine relevant information to surface via the interface based in part on the total time associated with the respective clinical visits.

In some examples, the service provider may determine which of the potential diagnoses, medications, gaps in care, and/or clinical recommendations to present to the medical provider based on a score associated therewith and/or other ranking structure. In some examples, the score and/or ranking structure may be determined based on severity of the potential diagnoses, medications, gaps in care, and/or clinical recommendations. In some examples, the score and/or ranking structure may be determined based on a time associated with the potential diagnoses, medications, gaps in care, and/or clinical recommendations. For example, a prescription that is overdue by 20 days may be ranked above (e.g., higher score or lower score) than a prescription that is overdue by 1 day. For another example, a prescription that is critical to a patient's health (e.g., immunosuppressant, injectable therapy, etc.) may be ranked above a prescription that is elective for the patient.

Though described as an interface for providing relevant information to a medical provider during a clinical visit, the interface may additionally or alternatively be accessed by a medical provider at a time outside of a clinical visit. In some examples, the medical provider may access the interface to review member data, process medications, submit claims for service, submit referrals, or the like. For example, the medical provider may access the interface to submit a referral for the patient to undergo a medical procedure outside of the clinical visit. In some examples, the medical provider may access the interface via an instance of the application on a computing device associated with the medical provider and/or via a website.

The techniques described herein improve a user interface of a computing device by providing real-time and/or near real-time information about a patient to a medical provider. The information provided via the interface may not otherwise be available to the medical provider but for the service provider's unique position in the medical industry. The information may include patient-specific information and, in some instances, may be ranked in order of importance, such as to enable the medical provider to quickly and efficiently conduct a clinical visit. Moreover, the information may enable a medical provider to provide a substantially improved level of care to the patient.

Additionally, the techniques described herein improve performance of one or more computing devices by reducing an amount of time a medical provider would need to access the interface. For instance, by surfacing the relevant information to the medical provider during the clinical visit, the service provider may decrease a time required for the clinical visit. Because the medical provider has access to more information, the time of use of the computing device associated with the interface may be decreased, thereby reducing an amount of processing and battery power required by computing device. Furthermore, the interface may provide a means by which the medical provider may quickly and easily submit information to the service provider, such as referrals, claims for payment, and the like. Because the medical provider may submit bundled information via the interface (in lieu of separate submissions and additional information required therewith), the techniques described herein may reduce a total amount of network resources required, thereby providing additional network resources for other computing devices.

These and other aspects are described further below with reference to the accompanying drawings. The drawings are merely example implementations and should not be construed to limit the scope of the claims. For example, while examples are illustrated in the context of a user interface for a mobile device, the techniques may be implemented using any computing device and the user interface may be adapted to the size, shape, and configuration of the particular computing device. Also, while many of the examples are given in the context of a clinical visit, the techniques described herein may also be applied to any other context associated with providing medical care, such as record reviews, submitting referrals, and the like.

### Example System Architecture

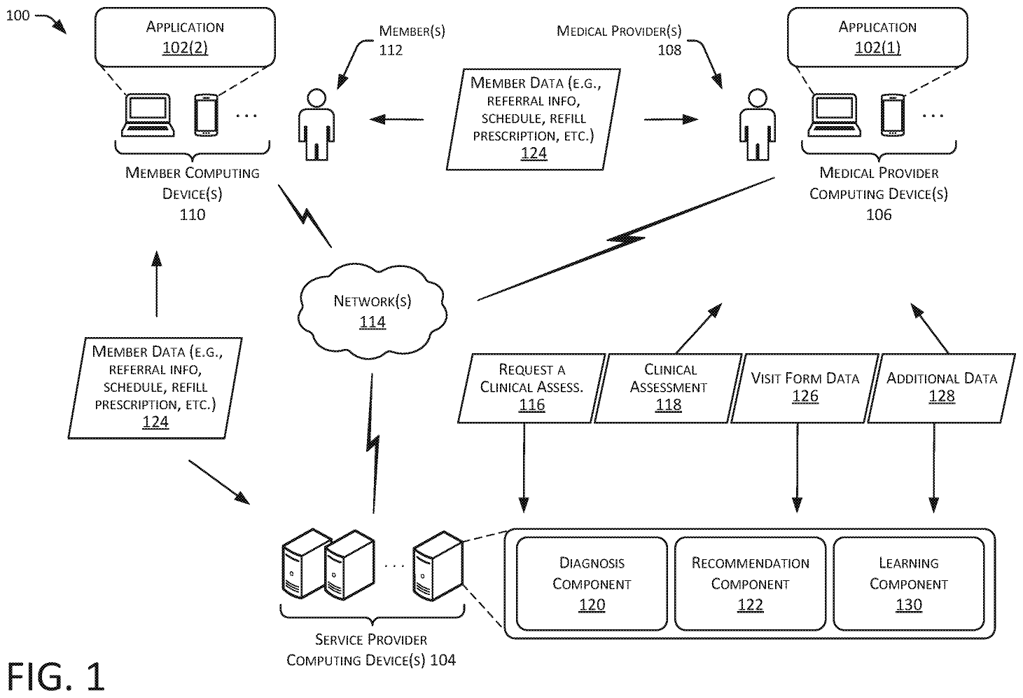

FIG. 1 is a schematic view of an example system 100 usable to implement the techniques described herein to provide relevant information via an instance of an application 102 via the system 100. In some examples, the system 100 may include a one or more service provider computing devices 104 (e.g., service provider 104) configured to manage the application 102, such as to provide the relevant information to one or more medical provider computing devices 106 (e.g., provider device(s) 106) associated with one or more medical providers 108. In various examples, the service provider computing device(s) 104 may additionally configured to provide information to one or more member computing devices 110 associated with one or more members 110. In various examples, the provider device(s) 106 may include a first instance of the application 102(1) and the member device(s) 110 may include a second instance of the application 102(2), to facilitate information flow to the medical provider(s) 108 and the member(s) 112.

Each of the provider device(s) 106 and the member device(s) 110 include one or more processors and memory storing computer executable instructions to implement the functionality discussed herein attributable to the respective computing devices. In some examples, the provider device(s) 106 and the member device(s) 110 may include desktop computers, laptop computers, tablet computers, mobile devices (e.g., smart phones or other cellular or mobile phones, mobile gaming devices, portable media devices, etc.), or other suitable computing devices. The provider device(s) 106 and the member device(s) 110 may execute one or more client applications, such as a web browser (e.g., Microsoft Windows Internet Explorer, Mozilla Firefox, Apple Safari, Google Chrome, Opera, etc.) or a native or special-purpose client application (e.g., service provider application, etc.), to access and view information provided by the service provider computing device(s) 104 over network 114.

In various examples, the provider device(s) 106 may include a single computing device. For example, a small medical office may include a single computing device for managing patient services, such as to prepare for a clinical visit, prepare a clinical visit, submit referrals, submit insurance claims, and the like. In some examples, the provider device(s) 106 may include one or more other computing devices, any or all of which may include one or more processors and memory storing computer executable instructions to implement the functionality described herein. For example, a larger medical office may include a first set of computing devices associated with medical office staff, such as schedule clinical visits, prepare for clinical visits, submit insurance claims, and the like, and a second set of computing devices associated with the medical provider, such as for conducting clinical visits, submitting referrals, and the like.

In some examples, the first instance of the application 102(1) may include one or more APIs configured to provide the medical provider(s) 108 functionalities within the first instance of the application 102(1) that differ from the second instance of the application 102(2). In some examples, the API(s) may include an enterprise client that enables multiple agents associated with the medical provider(s) 108 to access and respond to requests for information from the service provider computing device(s) 104 over the network 114.

Network 114 may represent a network or collection of networks (such as the Internet, a corporate intranet, a virtual private network (VPN), a local area network (LAN), a wireless local area network (WLAN), a cellular network, a wide area network (WAN), a metropolitan area network (MAN), or a combination of two or more such networks) over which the provider device(s) 106 and the member device(s) 110 may access the service provider computing device(s) 104 and/or communicate with one another.

The service provider computing device(s) 104 may include one or more servers or other computing devices, any or all of which may include one or more processors and memory storing computer executable instructions to implement the functionality discussed herein attributable to the medical insurance system or information platform. In various examples, the service provider computing device(s) 104 may store data associated with the member(s) 110 (e.g., member data) and the medical provider(s) 106 (e.g., medical provider data), such as in a member account or provider account, respectively. The member data may include demographic information (e.g., age, gender, ethnicity, race, occupation, marital status, etc.), member characteristics (e.g., hair color, eye color, shoe size, prosthetics, orthotics, etc.), medical history (e.g., previous diagnoses, medical procedures, surgeries, family medical history, etc.), laboratory results (e.g., glucose, cholesterol, etc.), medical test results (e.g., Echo stress test result, EKG, etc.), member location information (e.g., home address, work address, etc.), pharmacological information (e.g., prescriptions, prescription fill information (e.g., last fill, expirations, etc.), preferred pharmacy, etc.), and the like. The medical provider data may include provider location information (e.g., office locations, home address of medical provider, etc.), names and credentials of medical providers associated with a medical office and/or location, clinical visit times (e.g., average time, preferred (target) time, longest visit, shortest visit, etc.), insurance billing history, procedural history, procedural and/or practice specialties, provider quality metric (e.g., based on quality of service (e.g., based on feedback from members, professional organizations, awards earned, etc.), claim submissions (e.g., submitted on time with limited errors, etc.), percentage of patients associated with the service provider, use and management of the service provider application (e.g., clinical assessments, submitting referrals, etc.), ease of scheduling, delays associated with scheduling, etc.), and other information associated with the medical provider.

FIG. 1 illustrates an example in which a medical provider 108 may submit a request for a clinical assessment 116 to the service provider computing device(s) 104 via the first instance of the application 102(1). Though illustrated as a request for a clinical assessment 116, the techniques described herein may also be applied to any other type of request submitted to the service provider computing device(s) 104, such as inquiries about insurance bills, payment information, laboratory results, and the like. In various examples, the medical provider 108 may launch the first instance of the application 102(1) on the provider device 106, input data associated with a clinical visit (e.g., identifying information associated with the member (e.g., name, identifier, date of birth, etc.) and/or information associated with the appointment (e.g., provider, location, date, time, etc.)), and send the request for a clinical assessment to the service provider 104.

In various examples, responsive to receiving the request for a clinical assessment 116 the service provider 104 may generate a clinical assessment 118 to be surfaced to the medical provider 108 via the first instance of the application 102(1). In some examples, the service provider computing device(s) 104 may access member data and/or medical provider data to generate the clinical assessment 118, based on the information provided in the request for a clinical assessment 116.

In various examples, the clinical assessment 118 may include one or more potential diagnoses associated with the patient. A potential diagnosis may represent a condition that a member 112 may have, as determined by the service provider 104 based on member data. In at least one example, a diagnosis component 120 of the service provider computing device(s) 104 may be configured to determine the one or more potential diagnoses associated with the patient. In some examples, the diagnosis component 120 may utilize one or more machine learning techniques to determine the one or more diagnoses associated with the patient. In such examples, a machine learning model may be trained to identify one or more diagnoses and/or probabilities associated therewith based on training data including medical data corresponding to diagnoses.

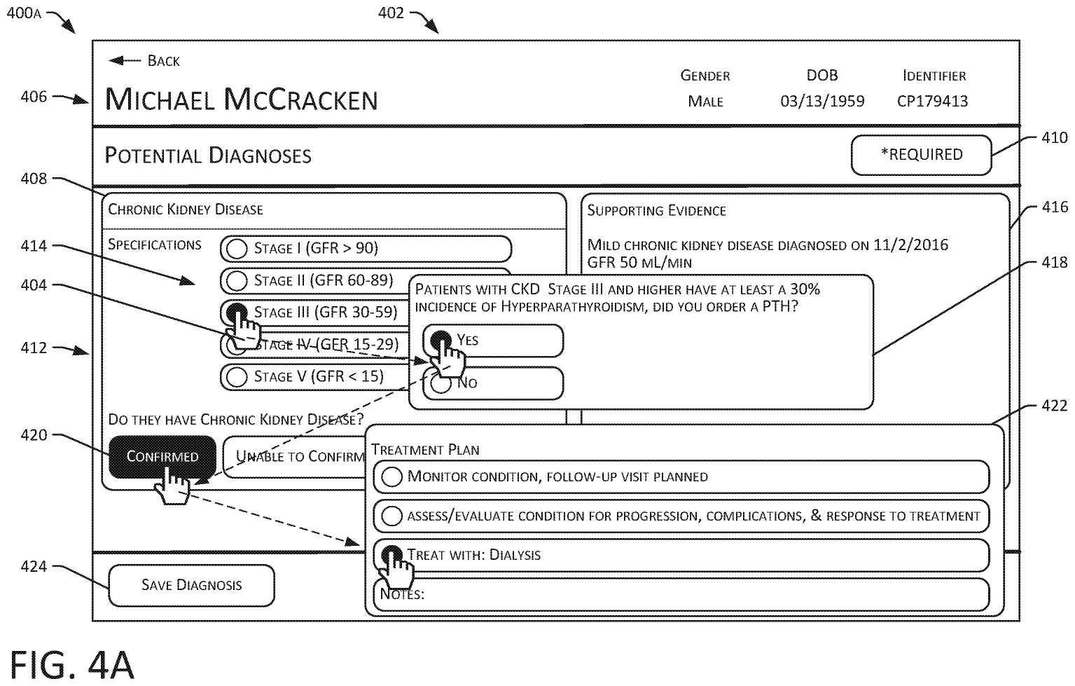

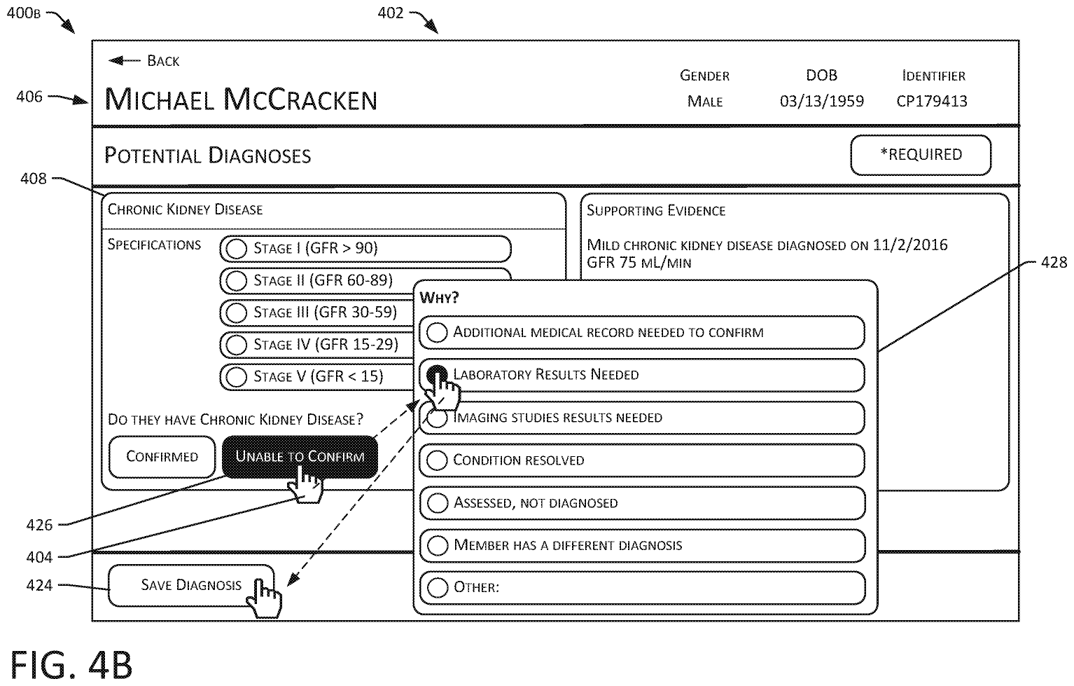

As will be discussed in further detail below with regard to FIGS. 4A and 4B, the clinical assessment may include a means by which the medical provider 108 may confirm (or not) a potential diagnosis. In some examples, responsive to receiving an indication that a potential diagnosis is confirmed, the service provider computing device 104 may update member data associated with the member 112 to reflect the diagnosis. In some examples, responsive to receiving an indication that the medical provider 108 is unable to confirm the potential diagnosis, the service provider 104 may request additional information from the medical provider 108, such as to provide a justification for the inability to confirm the potential diagnosis. In such examples, the diagnosis component 120 may process the additional. In some examples, the additional information may be used to further train the machine learned model with regard to potential diagnosis determination.

In various examples, the one or more potential diagnoses may be ranked by the diagnosis component 120. The ranking may be based on a determined level of severity of the diagnosis (e.g., terminal, minor condition, etc.), a probability that the member 112 suffers from the associated condition (e.g., determined based on member data by the service provider 104, the machine learning model, etc.). In various examples, the one or more potential diagnoses provided in the clinical assessment 118 may be based in part on the ranking (e.g., the level of severity, probability, etc.). In some examples, the inclusion of a potential diagnosis in the clinical assessment may be based on a probability associated therewith meeting or exceeding a threshold probability.

In some examples, a number or inclusion of one or more potential diagnoses provided in the clinical assessment 118 may be based on the ranking associated with each potential diagnosis. For example, the diagnosis component 120 may determine to include the highest ranked or the two highest ranked potential diagnoses. In some examples, a number or inclusion of one or more potential diagnoses provided in the clinical assessment 118 may be based on a time associated with an assessment of each potential diagnosis In such examples, the diagnosis component 120 may determine a time associated with an assessment of each potential diagnosis and may determine whether to include the potential diagnosis based in part on the time. The assessment may include an estimated amount of time for a medical provider 108 to assess whether a member suffers from the associated condition, such as to be able to confirm the diagnosis. The amount of time may be based on the particular medical provider, such as included in the provider data, and/or it may include an average time for a plurality of medical providers to address the potential diagnosis. In some examples, the service provider computing device(s) 104 may apply a pre-determined amount of time (30 seconds, 1 minute, 2 minutes, etc.) to all potential diagnoses. In some examples, the diagnosis component 120 of the service provider computing device 104 may be configured to include each of the determined potential diagnoses in the clinical assessment 118, to maximize member awareness of potential conditions and to initiate a discourse between the medical provider 108 and the member 112.

In various examples, the clinical assessment 118 may additionally include medication information. A recommendation component 122 of the service provider computing device(s) 104 may be configured to determine the medication information based on the member data. The medication information some or all current prescriptions and/or past or expired prescriptions, current and/or previously consumed medications (e.g., over-the-counter medications, etc.) (collectively referred to herein as “medications”) associated with the member. In some examples, the medication information may include a prescriber associated with the prescriptions and/or relevant dates (e.g., fill date, expiration date, etc.).

In various examples, the medications provided in the clinical assessment 118 may be ranked in order of importance, such as from a high level of importance (e.g., life threatening if not taken) to a low level of importance (e.g., elective medications, over-the-counter medications, etc.). In various examples, a number of medications provided may be based on the ranking. In some examples, a predetermined number of medications (e.g., all up to 5 medications, top 10 ranked medications, etc.) may be provided via the clinical assessment 118. In various examples, the number of medications provided in the clinical assessment 118 may be determined based on a time associated with an assessment and/or discussion associated with each medication. In such examples, the recommendation component 122 may determine a time associated with each medication. In some examples, the time may include an average time to assess and/or discuss medications, such as based on information determined by the service provider computing device 104 (e.g., statistical information, learned information from the application 102, etc.).

In various examples, a recommendation component 122 of the service provider computing device(s) 104 may be configured to generate one or more notifications to be included in the clinical assessment. The notification(s) may include a recommendation to extend a prescription (e.g., from a 30-day prescription to a 100-day prescription, etc.), to inform the member 112 about generic drug availability, encourage the member to fill prescriptions on time and to adhere to the prescription schedules, and the like. In various examples, the notifications may include a means by which the medical provider 108 may renew and/or modify a prescription, order a new prescription, and/or initiate a discussion about the prescription. For example, based on a first notification that a prescription is 100 days overdue, a medical provider 108 may ask the member, during the clinical visit, why they have not filled the prescription. Alternatively, based on a second notification about generic drug availability, the medical provider 108 may suggest a less expensive alternative to the original prescription. In various examples, responsive to receiving input from the medical provider 108 with regard to a renewal, modification, new prescription, and/or discussion about a prescription, the service provider may update the member data, and/or process approval for any renewed, modified, and/or new prescription.

In various examples, the recommendation component 122 of the service provider computing device(s) 104 may be configured to determine one or more gaps in care associated with the member care. A gap in care may include a screening, procedure, test, surgery, consult, or the like that the member should undergo. In some examples, the gap in care may be determined based on recommended care guidelines (e.g., health maintenance guidelines, recommended health screenings, etc.), such as those published by the center for disease control or other health organization. In some examples, the recommendation component 122 may determine the gap(s) in care based on the member data and may include the gap(s) in care in the clinical assessment 118.

Similar to that described above with regard to potential diagnoses, the particular gaps in care provided in the clinical assessment 118 may be based on a ranking associated therewith. In some examples, the ranking may be based on a level of severity associated with the gap in care not being addressed. In such examples, the recommendation component 122 may be configured to determine the level of severity based at least in part on a statistical analysis. In various examples, the gaps in care and/or a number of gaps in care provided in the clinical assessment 118 may include those associated with a level of severity above a threshold severity. In some examples, a number of gaps in care included in the clinical assessment 118 may include a pre-determined number (e.g., 1 gap in care, 2 gaps in care, etc.). In some examples, the number of gaps in care and/or the pre-determined number of gaps in care included in the clinical assessment 118 may be determined based on a time associated with processing the gaps in care. In such examples, the time associated with processing may include a time to determine whether a procedure is necessary (e.g., completed but not included in member data, member not eligible, etc.), to discuss a procedure associated with a gap in care, and/or to submit a referral for the procedure.

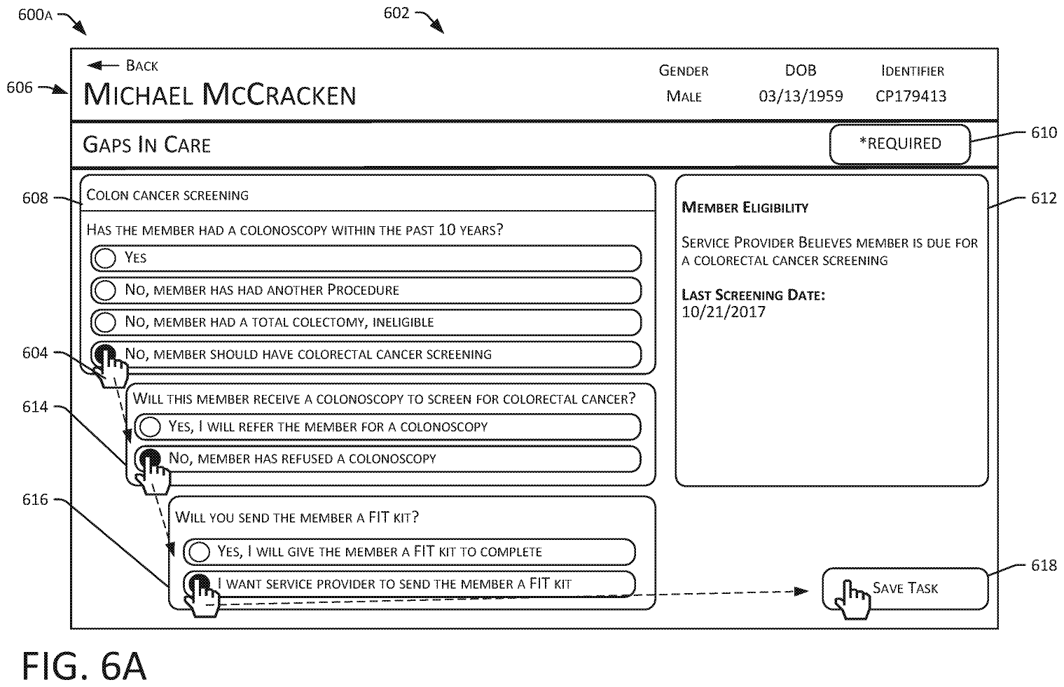

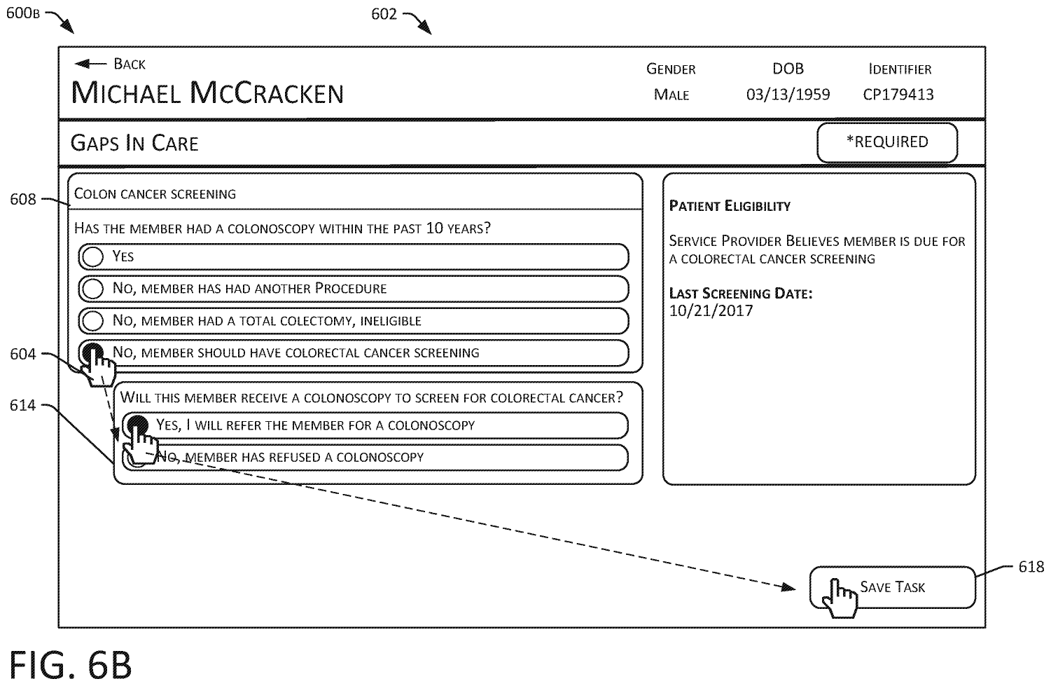

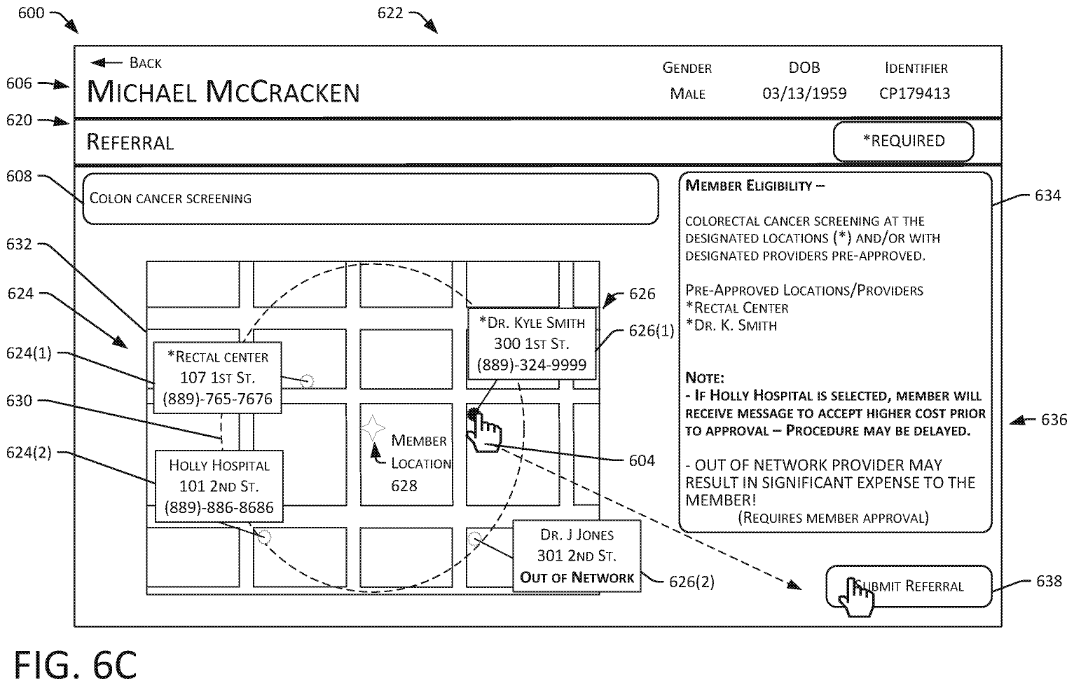

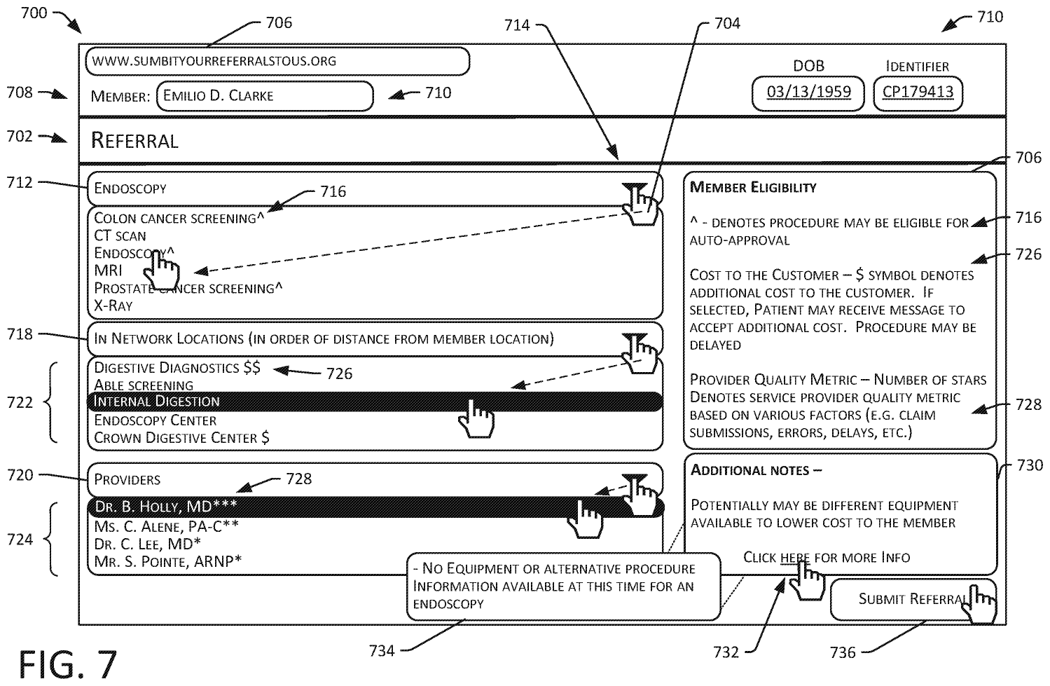

In some examples, the clinical assessment 118 may include a means by which the medical provider 108 may submit a referral to address the gap(s) in care. As will be discussed in greater detail below with regard to FIGS. 6B, 6C, and 7, responsive to receiving an indication that the medical provider 108 intends to submit a referral, the recommendation component 122 may identify one or more locations and/or providers available to provide a service associated with the referral. In some examples, the locations and/or medical providers may be determined based in part on a location associated therewith, a member location, a quality metric associated with the location and/or medical provider, a cost associated with the service at various locations and/or by various providers, insurance coverage approval, association with network (e.g., in network or out of network), and the like. In various examples, the recommendation component 122 of the service provider computing device(s) 104 may cause the identified location(s) and/or providers and information associated therewith to be surfaced to the medical provider via the first instance of the application 102(1). Additionally or alternatively the recommendation component 122 of the service provider computing device 104 may cause the identified location(s) and/or providers and information associated therewith to be surfaced to the member 112 via the second instance of the application 102(2), such as in member data 124. In some examples, the system may be configured to share select member data 124, such as that including the referral information, between a medical provider computing device 106 and a member device 110.

In various examples, the recommendation component 122 of the service provider computing device(s) 104 may be configured to generate one or more clinical recommendations for the medical provider 108. In some examples, the recommendation component 122 may include the clinical recommendation(s) in the clinical assessment. The clinical recommendation(s) may include information to inform medical decisions, such as information associated with a member diagnosis (e.g., known diagnosis), a potential treatment for the member 112, studies published regarding a diagnosis or medical condition associated with the member 112, and the like. In various examples, the clinical assessment 118 may include supporting documentation for the clinical recommendations, such a diagnosis, medications, and/or lab results that support the clinical recommendation. In some examples, the supporting documentation may include clinical guidelines for treating a particular diagnosis or condition.

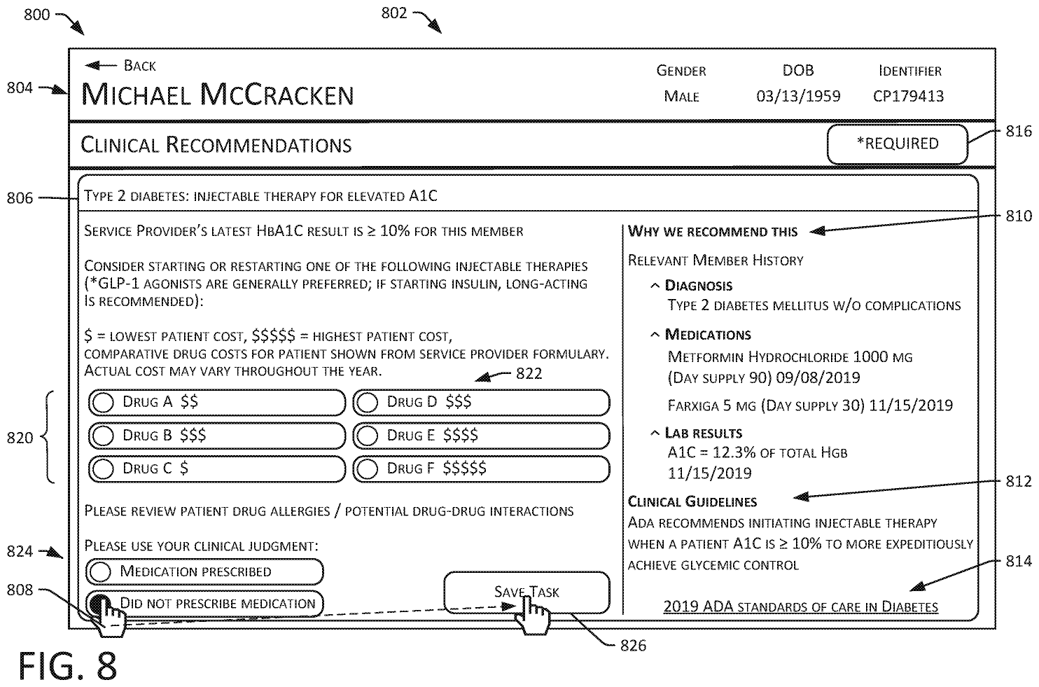

As will be discussed in greater detail below with regard to FIG. 8, the clinical assessment 118 may include a request for input regarding the clinical recommendation(s), such as whether the medical provider will act on a particular clinical recommendation. In various examples, the recommendation component 122 may be configured to rank the clinical recommendation(s), such as based on severity and/or importance to the member 112. In some examples, the clinical assessment 118 may include a pre-determined number of clinical recommendations (e.g., 1, 2, etc.). In some examples, the recommendation component 122 may determine a number of clinical recommendations to include in the clinical assessment 118, such as based on a threshold severity and/or importance to the member. For example, a clinical recommendation that may significantly improve patient care may be assessed a high importance (e.g., 10 on a scale of 1 to 10) and a clinical recommendation that may only minorly impact the patient may be assessed a low importance (e.g., 1 on the scale of 1 to 10). The clinical recommendation with high importance (e.g., meets or exceeds a threshold importance, highest importance of ranked clinical recommendations, etc.) may be included in the clinical assessment 118, while the clinical recommendation with the low importance (e.g., below a threshold) may not be included.

As discussed above, the clinical assessment 118 may provide a means by which the service provider 104 may request information from the medical provider and/or a means by which the medical provider 108 may provide clinical assessment data 126 to the service provider 104. The clinical assessment data 126 may include, but is not limited to, a confirmation of a potential diagnosis, reason for not confirming the potential diagnosis, medication modifications, renewals, new prescriptions, referrals and/or responses to gaps in care, responses to clinical recommendations, and/or other information pertinent to the clinical visit between the medical provider 108 and the member 112.

In various examples, the application 102 may additionally provide a means by which the service provider 104 and the medical provider 108 may be share additional data 128. The additional data 128 may include insurance claims, payment information, scheduling information, member status notifications (e.g., member admitted to emergency room, released from a hospital, etc.). In various examples, the additional data 128 may include a reminder to the medical provider 108 to use the application 102(1) during the clinical visit. In such examples, the reminder may include a short-message system message, an electronic mail message, a phone call, or the like. In various examples, the reminder may be surfaced on a display associated with the medical provider computing device(s) 106, such as via a push notification.

In various examples, the service provider computing device(s) 104 may include a learning component 130. In such examples, the learning component 130 may train one or more machine learned models associated with the diagnosis component 120, the recommendation component 122, and/or other components of the service provider computing device(s) 104. For example, as described above, the learning component 130 may train a machine learning model to determine one or more potential diagnoses associated with a patient. In some examples, the learning component 130 may train a machine learning model of the recommendation component 122 to determine one or more locations and/or a provider that may be pre-approved by the service provider 104. In such examples, the determination for pre-approval may be provided to the medical provider 108 to inform a decision regarding a referral. In some examples, the machine learned model may be trained utilizing training data associated with previously approved procedures, the training data including a member risk score, information associated with a provider and/or location in the referral, the medical provider 108 submitting the referral, a procedure associated with the referral, and/or other data considered in a manual review for approval.

Additionally or alternatively, the learning component 130 may train one or more machine learned models associated with the recommendation component 122 to determine one or more clinical recommendations. Although specifically enumerated examples of machine learning models and outputs thereof are described herein, these are provided for illustrative purposes and are not intended to be so limiting. Other examples of machine learning models configured to provide different outputs, such as likelihood of receiving payment, recovery probabilities, and the like are contemplated herein. In such examples, the service provider computing devices 104 may be configured to provide the outputs to the medical provider 108 and/or the member 112.

In various examples, the service provider computing device 104 may be configured to provide select member data 124 (e.g., member data 124 authorized to be transmitted to the member 112 and/or medical provider 108, based at least in part on rules and/or regulations associated with member data) directly to the member 112 via the second instance of the application 102(2). As discussed above, the member data 124 may include referral information. In some examples, the member data 124 may include schedule information (e.g., scheduled clinical visits, screenings, etc.), prescription information (e.g., refills ordered, refills pending authorization, new prescriptions, etc.), insurance data, and any other information pertinent to the member 112 regarding the medical care thereof.

In some examples, the member 112 may send member data 124 to the service provider computing device(s) 104. In some examples, the member data 124 sent from the member 112 to the service provider 104 may include initial member data, such as that provided to set up an account with the service provider 104. In some examples, the member data 124 sent from the member 112 to the service provider 104 may include updated information regarding the member 112, such as care provided outside of a network associated with the service provider, schedule updates, or the like.

### Example User Interfaces

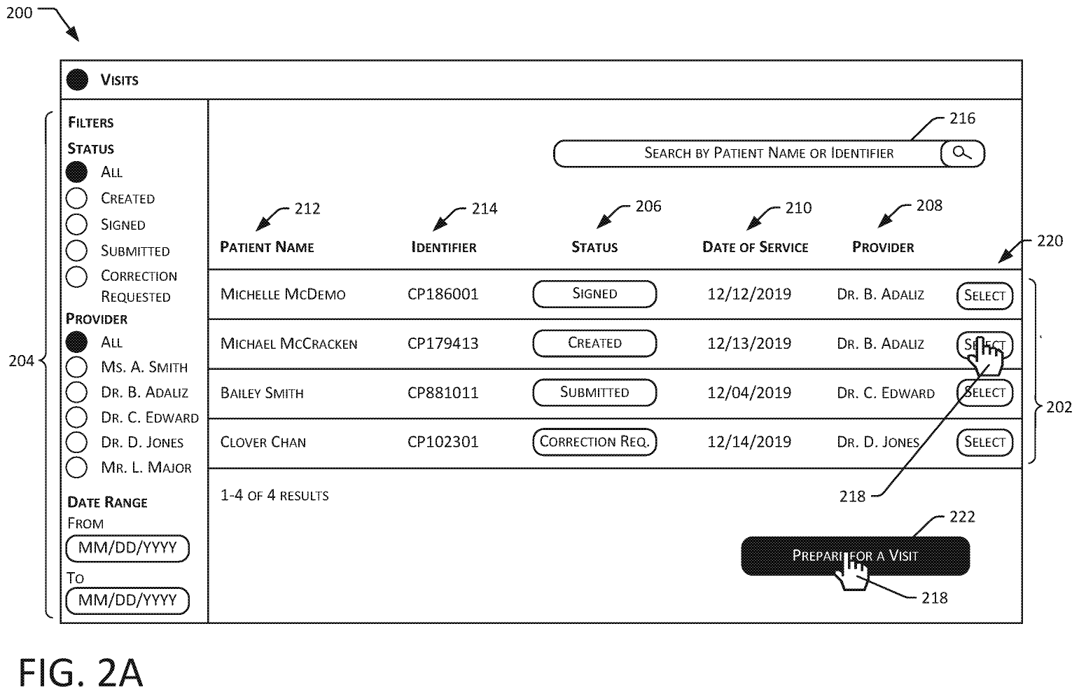

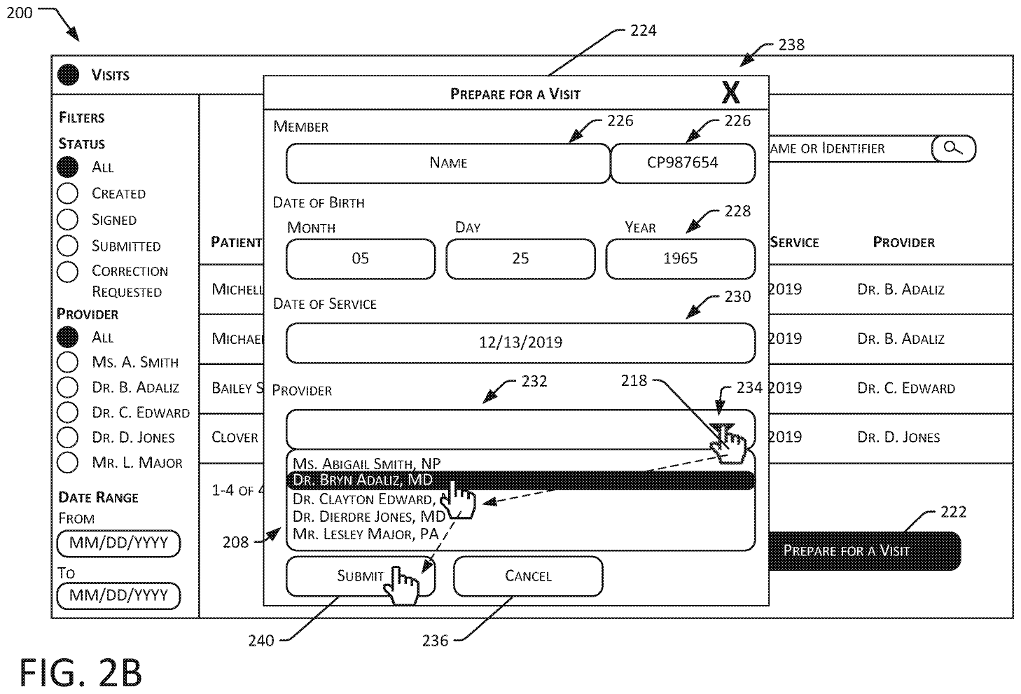

FIG. 2A—FIG. 8 are schematic views showing example user interfaces that are usable to implement the techniques described herein for providing relevant information to assist in providing effective health care management for a member. The interfaces may be generated by one or more computing device of a service provider (e.g., service provider computing device(s) 104) and transmitted to one or more medical provider computing devices (e.g., provider device(s) 106) and/or one or more member computing devices (e.g., member device(s) 110) for presentation. Additionally or alternatively, the interfaces may be generated by the provider device(s) and/or the member device(s) based at least in part on instructions received from the service provider communication device(s). As discussed above, the interfaces described in this section may, but need not, be implemented in the context of the system 100.

FIGS. 2A and 2B illustrate an example interface in which clinical assessments may be reviewed and generated. FIG. 2A illustrates an example interface 200 in which previously generated clinical assessments 202 may be reviewed. The clinical assessments 202 may include completed clinical assessments and/or data submitted by a medical provider and/or a member during a clinical visit, such as that described herein.

In various examples, the interface 200 may present the clinical assessments 202 based on one or more filters 204. In the illustrative example, the filter(s) 204 include a status of the application (e.g., status 206), a provider 208, and a date range associated with dates of service 210. In other examples, the filter(s) 204 may include other information, associated with clinical assessments, such as clinical assessments including a confirmed diagnosis, a medication modification, a new prescription, or the like.

In various examples, the clinical assessments 202 presented on the interface 200 may be ordered, such as based on an alphabetical order by patient name 212, based on an identifier 214, by the provider 208, or the like. In some examples, the clinical assessments 202 presented on the interface 200 may be presented in a random order.

In various examples, the interface 200 may include a search function 216, in which a medical provider 218 (or associate thereof) may search for a particular patient name 212 or patient identifier 214. In various examples, the medical provider 218 may access a particular clinical assessment 202 by selecting a first selectable option 220 associated with the particular clinical assessment 202. In such examples, the medical provider 218 may be able to modify the clinical assessment and/or update a status 206 associated therewith.

In various examples, the medical provider 218 may generate a new clinical assessment 202 by selecting a second selectable option 222. Though illustrated in FIG. 2A with a corresponding label to “PREPARE FOR A VISIT,” this is merely for illustrative purposes, and any other label associated with generating a clinical assessment 202 is contemplated herein, such as “NEW,” “NEW CLINICAL ASSESSMENT,” “ADD CLINICAL ASSESSMENT,” or the like.

FIG. 2B illustrates the example interface 200 configured for generating a new clinical assessment, such as by selecting the second selectable option 222. As discussed herein, the new clinical assessment may be generated for an upcoming clinical visit between the medical provider 218 and a member.

In the illustrative example, responsive to selecting the selectable option 222, a window 224 may surface via the interface 200. The window 224 may include data fields 226, 228, 230, and 232 for the medical provider 218 to input relevant data about an upcoming clinical visit. In other examples, a new page associated with the interface 200 may launch, such as via the application or a website. In such examples, the new page may include data fields the same and/or similar to data fields 226, 228, 230, and 232.

In the illustrative example, a first data field 226 includes a member name and identifier. In various examples, the identifier may include an alphanumeric identifier, a symbol, or other indicator of a particular identifier associated with a particular member. In other examples, the first data field 226 may include either the member name or the identifier. As illustrated, the second data field 228 may include a date of birth associated with the member. In other examples, the window may include additional or alternative data fields to identify the particular member.

In the illustrative example, the third data field 230 includes a date of service associated with the clinical visit associated with the new clinical assessment, such as a date that the clinical visit is scheduled. The fourth data field 232 includes a drop-down menu option 234 for the medical provider 218 to select a provider 208 associated with the clinical visit. In other examples, the fourth data field 232 may include a means by which the medical provider 218 may type in the provider 208 associated with the clinical visit. In some examples, the fourth data field 232 may include an auto-fill option, such that the fourth data field 232 may auto-fill based on an order in which letters are typed into the data field.

In various examples, the window 224 may include a means by which the creation of the new clinical assessment may be canceled, such as by a third selectable option 236 to cancel and/or a fourth selectable option 238 (illustrated as an X). In various examples, one or both of the third selectable option or the fourth selectable option 238 may surface an option for the medical provider 218 to save changes. In such examples, the data input into the window 224 may be saved to complete at a later time. In some examples, the interface 200 may provide an indication of a partially complete (e.g., not yet submitted) request for a new clinical assessment.

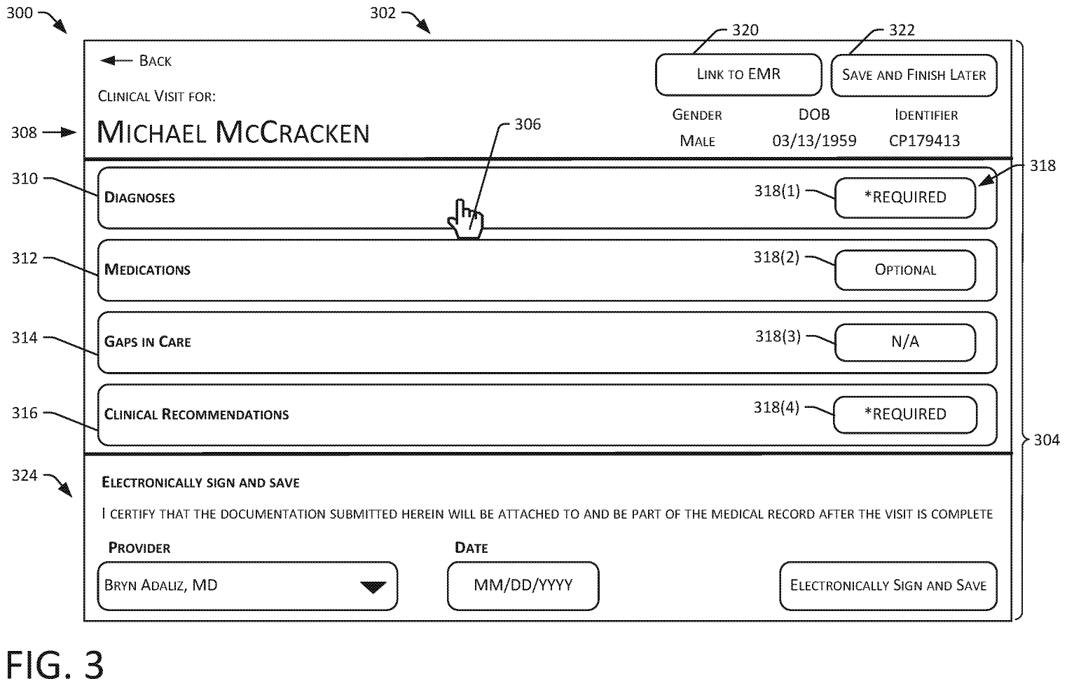

In various examples, responsive to the medical provider selecting a fifth selectable option 240 to submit the request to generate the new clinical assessment, the service provider may generate a clinical assessment, such as that illustrated in FIGS. 3-6C and 8.

FIG. 3 illustrates an example interface 300 associated with a main page 302 (e.g., home page, etc.) associated with a clinical assessment 304, such as clinical assessment 118 and 202, generated by a service provider. In various examples, a medical provider 306, such as medical provider 108 and 208, may access the clinical assessment 304 by selecting the first selectable option 220 depicted in FIG. 2A. In such an example, an application associated with the service provider, such as application 102, may cause the main page 302 of the clinical assessment 304 to launch (e.g., surface on a display of a provider device) In some examples, the main page 302 associated with the clinical assessment 304 may automatically launch on the application based on an indication of selection to submit a request to generate the clinical assessment, such as the fifth selectable option 240 to request a new clinical assessment.

In the illustrative example, the main page 302 includes member data 308, such as a name, gender, date of birth, and identifier. In other examples, additional or alternative member data 308 may be included, such as age, ethnicity, or the like. As illustrated, the main page 302 may include a diagnoses selectable option 310, a medications selectable option 312, a gaps in care selectable option 314, and a clinical recommendations selectable option 316. Responsive to selection of one of the diagnoses selectable option 310, the medications selectable option 312, the gaps in care selectable option 314, or the clinical recommendations selectable option 316, a respective page may launch via the application, such as that depicted in FIGS. 4, 4B, 5, 6A-6C, and 8.

In various examples, one or more of the diagnoses selectable option 310, the medications selectable option 312, the gaps in care selectable option 314, or the clinical recommendations selectable option 316 may be included in the main page 302. In various examples, the service provider may determine a number of diagnoses, medications, gaps in care, and/or clinical recommendations for the medical provider 306 to address (e.g., review information, discuss with member, etc.). In some examples, based on a determination that the number of diagnoses, medications, gaps in care, and/or clinical recommendations is at least one, the diagnoses selectable option 310, the medications selectable option 312, the gaps in care selectable option 314, or the clinical recommendations selectable option 316 may be included on the main page 302.

In various examples, based on a determination that the number of diagnoses, medications, gaps in care, and/or clinical recommendations is at least one, the respective selectable option 310, 312, 314, and/or 316 may include a label 318. The label 318 may indicate whether a review of the diagnoses, medication, gaps in care, and/or clinical recommendations is required, optional, or not applicable. In the illustrative example, a first label 318(1) associated with the diagnoses is required to review, a second label 318(2) associated with medication is optional to review, a third label 318(3) associated with gaps in care is not applicable (N/A), and a fourth label 318(4) associated with clinical recommendations is required. In other examples, one or all of the diagnoses, medications, gaps in care, and clinical recommendations may be required to review, optional to review, or not applicable.

In some examples, the service provider may determine that the review of one or more of the diagnoses, medications, gaps in care, and/or clinical recommendations is optional based on a total time allocated for the clinical assessment and/or a level of importance associated with each of the diagnoses, the medications the gaps in care, and/or the clinical recommendations. The total time may include a time associated with a particular medical provider 306, such as stored in data associated with the particular medical provider 306. For example, a service provider may indicate that a clinical assessment should be a maximum of 7 minutes. Based on the total time of 7 minutes, the service provider may determine a time and/or ranking (e.g., level of importance based on severity, probability, etc.) associated with each of the diagnoses, medications, gaps in care, and/or clinical recommendations, and may determine that one or more of the diagnoses, medications, gaps in care, and/or clinical recommendations are optional.

In some examples, a determination that the review of one or more of the diagnoses, the medications, the gaps in care, and the clinical recommendations is optional may be based on the ranking (e.g., level of severity, probability) associated therewith. In some examples, based on a determination that the level of severity and/or the probability associated with the one or more of the diagnoses, the medications, the gaps in care, and the clinical recommendations is below a first threshold level of severity or probability, the service provider may determine that it a review thereof is optional. In some examples, based on a determination that the ranking is below a second threshold, the service provider may determine that the review is not applicable.

As illustrated in FIG. 3, the main page 302 may include a link to EMR (electronic medical record) selectable option 320 and a save and finish later selectable option 322. In other examples, the main page 302 may include one or both of the selectable options 320 and 322. In some examples, the link to EMR selectable option 320 provide a means by which the medical provider 306 may access an electronic medical record associated with the member. In some examples, selection of the link to EMR selectable option 320 may cause the member data submitted via the clinical assessment 304 to be automatically uploaded to the EMR associated with the member. In some examples, the main page 302 may include additional or alternative selectable options, such as a link to a relevant website (e.g., Center for Disease Control, American Diabetes Association, etc.). In some examples, the alternative selectable options may be determined based on member data, such as a current diagnosis or condition associated with the member.

In the illustrative example shown in FIG. 3, the main page 302 may include a signature section 324. The signature section 324 may provide a means by which the medical provider 306 may electronically sign the clinical assessment 304 and save data associated therewith. In various examples, the signature section may provide a means by which the medical provider 306 may submit the clinical assessment 304 to the service provider. In various examples, based at least in part on submission of the clinical assessment 304, the service provider may update member data and/or process an insurance claim associated with the clinical visit. In some examples, the service provider may additionally process an approval for a referral submitted with the clinical assessment 304, such as via the gaps in care section, as will be discussed below with regard to FIGS. 6B and 6C.

FIGS. 4A and 4B illustrate example interfaces associated with a diagnosis page 402 of a clinical assessment, such as clinical assessment 118 and 304. FIG. 4A illustrates an example interface 400A in which a medical provider 404 may confirm a diagnosis. The diagnosis page 402, similar to the main page 302 may include member data 406, such as a name, gender, date of birth, and identifier. In other examples, additional or alternative member data 406 may be included, such as age, ethnicity, or the like. The member data 406 may include the same or different data as member data 308 of the main page 302.

The diagnosis page 402 may include one or more potential diagnoses associated with the member. As discussed above, the service provider may determine the one or more potential diagnoses based at least in part on member data. In some examples, the potential diagnoses may be determined based at least in part on one or more machine learning models.

In some examples, the diagnosis page 402 may include one or more potential diagnoses 408 determined by the service provider. In some examples, the one or more potential diagnoses 408 may be presented on the diagnosis page 402 based on a ranking (e.g., level of severity, probability), such as that described above. In the illustrative example, the diagnosis page 402 includes a single potential diagnosis 408 with a label 410 indicating a requirement to address the diagnosis 408. In other examples, the diagnosis page 402 may not include any potential diagnoses, such as if the service provider determines that no potential diagnoses are associated with the member or that the member has previously been diagnosed with a condition and/or a diagnosis has previously been confirmed by the medical provider. In such examples, the label 410 may indicate that the requirement to address a potential diagnosis is not applicable.

In various examples, the potential diagnosis 408 may have associated therewith the requirement to address (e.g., label 410) based on a ranking associated with the potential diagnosis 408. As discussed above, the ranking may include a level of severity or a probability associated with the potential diagnosis 408. In some examples, the potential diagnosis 408 may be included on the diagnosis page 402 based on the level of severity being above a threshold level of severity. In some examples, the potential diagnosis 408 may be included on the diagnosis page 402 based on the probability that the potential diagnosis is associated with the member being above a threshold probability.

In various examples, the potential diagnosis 408 may include a request to confirm 412 that the member is associated with the potential diagnosis 408 (e.g., that the member has the condition associated therewith). In some examples, the request to confirm 412 may include additional information 414 regarding the potential diagnosis 408. For example, and as illustrated in FIG. 4A, the potential diagnosis 408 includes a chronic kidney disease. In various examples, the diagnosis page 402 may additionally include supporting evidence 416 associated with the potential diagnosis 408. In some examples, the service provider may determine the supporting evidence based on member data and/or applying member data to one or more machine learning models configured to output a potential diagnosis, a probability associated therewith, and/or one or more data points associated with the supporting evidence 416. For example, the supporting evidence 416 provides a glomerular filtration rate (GFR) (e.g., test result) associated with the member.

In the illustrative example, the supporting evidence 416 may be applicable to providing a confirmation of the potential diagnosis and/or submitting input regarding the additional information 414. For example, the GFR rate associated with the supporting evidence 416 may correspond to stage III of the chronic kidney disease. The medical provider 404 may thus select “STAGE III” of the additional information 414 during the diagnosis confirmation process.

In some examples, responsive to receiving input associated with the additional information, a first window 418 may surface on the diagnosis page 402. The first window 418 may include a recommendation, additional information about the disease, and/or a request for additional information from the medical provider 404. For example, responsive to the input of “STAGE III” kidney disease, the service provider may provide information about the disease, such as that patients with stage III or higher kidney disease have a higher incidence of hyperparathyroidism. The service provider may additional request information regarding whether the medical provider 404 has ordered a parathyroid hormone (PTH) test. In various examples, the service provider may surface a means by which the medical provider 404 may order an evaluation (e.g., laboratory, clinical, etc.) associated with the potential diagnosis 408 and/or stage (e.g., level, etc.) thereof.

In the illustrative example, based in part on addressing the additional information 414 associated with the potential diagnosis 408, the medical provider 404 may confirm the diagnosis, such as via the confirmation selectable option 420. In some examples, the service provider may receive an indication of confirmation responsive to selection of the confirmation selectable option 420. In various examples, the service provider may update the member data based on the confirmation.

In some examples, the service provider may cause a second window 422 to surface on the diagnosis page 402, the second window 422 including a request for information and/or additional information regarding the disease. In some examples, the additional information provided in the second window 422 may include resources for learning about the disease, issues associated with the disease to discuss with the member, medication information to prescribe and/or discuss with the member, treatment options, and the like. In the illustrative example, the second window 422 may include a request for information regarding a treatment plan for the member. In various examples, responsive to receiving an input regarding the treatment plan, the service provider may update the member data. Additionally or alternatively, responsive to receiving input via the second window 422, the service provider may initiate an insurance approval, such as for a treatment, prescription, or the like. For example, responsive to receiving a selection that the chronic kidney disease will be treated with dialysis, the service provider may process an approval for insurance to cover the treatment.

In various examples, the diagnosis page 402 may include a selectable option 424 to save the diagnosis information input via the diagnosis page 402. In various examples, responsive to selecting the selectable option 424, the confirmation selectable option 420, and/or other inputs associated with the potential diagnosis 408 (e.g., information input via the first window 418, the second window 422, etc.) may be sent to the service provider, such as message sent responsive to the input. For example, based on an indication of confirmation, a message may be sent from the application to the service provider indicating that the diagnosis is confirmed.

In various examples, the information associated with the diagnosis page 402 may be sent to the service provider based on a submission of the clinical assessment associated therewith. For example, based on an indication that a medical provider has signed and/or submitted a clinical assessment, such as that depicted in FIG. 3, the message may be sent to the service provider indicating the information input via the diagnosis page 402.

FIG. 4B illustrates an example interface 400B in which a medical provider 404 may be unable to confirm a diagnosis. In some examples, responsive to the medical provider 404 selecting an unable to confirm selectable option 426, the service provider may cause a third window 428 to surface via the diagnosis page 402. The third window 428 may include a request for additional information from the medical provider 404, such as to provide a justification for the inability to confirm the potential diagnosis 408. In some examples, the additional information may be sent to the service provider. Responsive to receiving the additional information, the service provider may store the additional information in the member data, initiate an approval for additional testing and/or medications, and/or cause the additional testing to be scheduled. Similar to that described above in FIG. 4B, the medical provider 404 may save the inputs associated with the diagnosis page 402 by selecting the selectable option 424 to save.

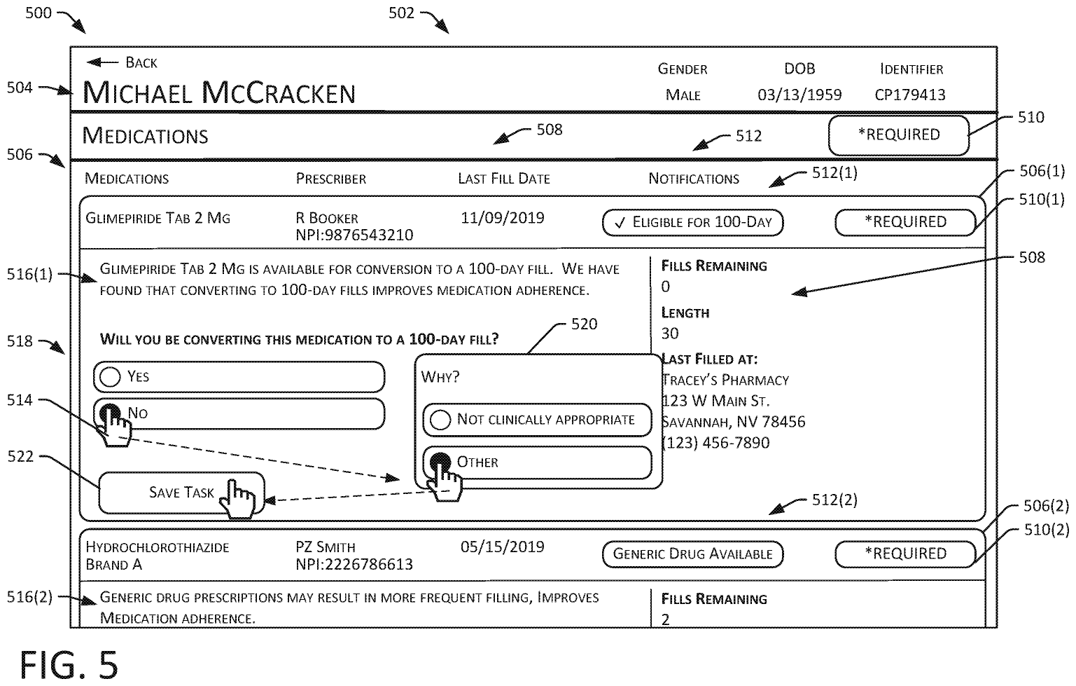

FIG. 5 illustrates an example interface 500 corresponding to a medications page 502 associated with a clinical assessment. The medications page 502, similar to the main page 302 may include member data 504, such as a name, gender, date of birth, and identifier. In other examples, additional or alternative member data 504 may be included, such as age, ethnicity, or the like. The member data 504 may include the same or different data as member data 308 of the main page 302.

As illustrated in FIG. 5, the medications page 502 may include one or more medications 506. The medications 506 may include medication data 508. In the illustrative example, the medication data 508 may include a name of a drug associated with the medication 506, a prescriber and prescriber identifier, a date in which the prescription was last filled, a location at which the prescription was filled, a number of fills remaining, and a length of the prescription (e.g., number of days associated with the prescription). In other examples, additional or alternative information may be included in the medication data 508, such as side effects associated with the medication 506, potential drug-drug interactions (e.g., do not prescribe this medication with medication B, the combination may result in elevated risk of heart attack), an indication of an overdue or soon to be due renewal, or the like.

In some examples, the service provider may determine the medication(s) 506 based on member data. In some examples, the one or more medications 506 may include current and/or active medications (e.g., associated with current prescriptions, based on an indication from the member that they are taking a medication, such as an over-the-counter medication, etc.). In the illustrative example, the medications page 502 includes a label 510 indicating a requirement to address the medications 506. Additionally, the individual medications 506, such as medication 506(1) and 506(2) may include labels 510(1) and 510(2) indicating a requirement to address. In other examples, the individual medications 506(1) and 506(2) may be optional to address.

In other examples, the medications page 502 may not include any medications, such as if the service provider determines that the member does not currently have any prescriptions and/or the member has indicated that they are not consuming any medications. In such examples, the label 510 may indicate that the requirement to address a potential diagnosis is not applicable. In some examples, the service provider may determine that the member has indicated a consumption of over-the-counter medications but does not have any prescriptions. In such an example, the label 510 may indicate that a review of the medications page 502 is optional or not applicable. As discussed above, the service provider may determine that the review of the medications page 502 is optional based on a total time allocated for the clinical assessment and/or a ranking associated with one or more other of the potential diagnoses, the gaps in care, and/or the clinical recommendations. For example, the service provider may determine that the medications review is optional based on a determination that an estimated time associated with a potential diagnosis may meet or exceed the total time allocated for the clinical assessment. In such an example, the potential diagnosis may be required, and the medications, gaps in care, and clinical recommendations may be optional.

In some examples, the medications page 502 may be required to review based on a ranking associated with a medication 506 (e.g., how important a medication 506(1) or 506(2) is to the member), a determination that there is a notification 512 associated with the medication 506(1) or 506(2), and/or any other issues for a medical provider 514, such as medical provider 108, 208, 306, and 404. In some examples, the medications 506(1) and/or 506(2) may be required to review and/or optional to review based on a threshold ranking (e.g., threshold level of importance). For example, the medications 506(1) and 506(2) may be required to review based on a determination that the respective rankings are above a first threshold. Based on a determination that one or more of the medications 506 are below the first threshold and/or above a second threshold, the medications may be optional to review. In some examples, based on a determination that the medications 506 are below the second threshold, a review of the medications 506 may not be required.

Additionally or alternatively, the review may be optional or not required based on a determination of a time associated with a review of the medication, a time associated with a review of the potential diagnoses, the gaps in care, and/or the clinical recommendations, and a total time allocated for the clinical assessment. In some examples, the service provider may rank an importance of each of the potential diagnoses, medications, gaps in care and clinical recommendations, and/or may determine a time associated with each. Based on the importance, the service provider may rank each of the potential diagnoses, medications, gaps in care and clinical recommendations within each group and against one another. Based on the overall ranking the service provider may determine whether the medications 506(1) and/or 506(2) are required to be reviewed. In some examples, based on the time associated with each of the potential diagnoses, medications, gaps in care and clinical recommendations and a total time allocated for the clinical assessment, the service provider may determine whether the medications 506(1) and/or 506(2) are required to be reviewed.

In some examples, the medications 506(1) and 506(2) presented via the medications page 502 may be determined based on the ranking associated therewith. In such examples, the medications 506(1) and 506(2) may be presented in a ranked order, such as based on an importance of the medication to the member (e.g., required for health, elective, etc.). For example, the glimepiride medication 506(1) may be more important to a member than the hydrochlorothiazide medication 506(2). In some examples, the medications 506(1) and 506(2) may be presented via the medication page 502 based on the labels 510(1) and 510(2) associated therewith (e.g., required or optional to address).

As discussed above, the requirement to address a medication 506 may be based on a determination that a notification 512 is associated therewith. The notification 512 may include an option to modify the associated medication. The notification 512 may include an eligibility for an extended prescription (e.g., from a 30-day prescription to a 100-day prescription, etc.), a generic drug availability, a member delay in filling the prescription, an overdue prescription, and the like. For example, a first notification 512(1) may include an eligibility to extend a prescription to a 100-day prescription and a second notification 512(2) may include an availability of a generic drug, which may include a less expensive option for the medication 506(2).

In some examples, the medications page 502 may include an explanation 516(1) associated with the first notification 512(1) and/or a second explanation 516(2) associated with the second notification 512(2). The explanations 516(1) and 516(2) may provide additional information about the respective notifications 512(1) and 512(2). As an illustrative example, the explanation 516(2) indicates to a medical provider 514, that the conversion to a 100-day fill may improve medication adherence.

In various examples, the medications page 502 may include a selectable option 518 associated with a request for a response regarding the notification 512(1). In the illustrative example, the medical provider 514 may indicate whether the medication will be converted to a 100-day fill. In some examples, responsive to receiving an indication that the medication will be modified, the service provider may update member data, process an approval for the modified medication, order the modified medication, update the prescription and/or send a message to the medical provider to update the prescription and/or order the modified medication as a separate task. In some examples, the.

In some examples, responsive to an indication that the medical provider 514 is not modifying the medication 506(1), the service provider may cause a window 520 to surface. In the example illustrated in FIG. 5, the window 520 may request a justification for not modifying the medication 506(1). In some examples, the window 520 may include additional information associated with other options available to the medical provider 514 and/or member, such as a different option for length of fill, different medication, or the like.

In the illustrative example, the medication page 502 may include a selectable option 522 to save the information about the medication 506(1) and/or 506(2). In some examples, the selectable option 522 may be associated with the medication page 502 as a whole. In such examples, responsive to selection of the selectable option 522, the application may save any data input via the medication page 502.

In various examples, responsive to selecting the selectable option 522 and/or other inputs associated with the medications 506 (e.g., information input via selectable option 518, the window 520, etc.) may be sent to the service provider, such as message sent responsive to the input. For example, based on an indication that the medication 506(1) will not be converted to a 100-day fill, a message may be sent from the application to the service provider indicating that the medication 506(1) will not be modified.

In various examples, the information associated with the medications page 502 may be sent to the service provider based on a submission of the clinical assessment associated therewith. For example, based on an indication that a medical provider has signed and/or submitted a clinical assessment, such as that depicted in FIG. 3, the message may be sent to the service provider indicating the information input via the medications page 502.

FIGS. 6A-6C illustrate example interfaces corresponding to a gaps in care page 602 associated with a clinical assessment, such as clinical assessment 118 and 304. FIG. 6A illustrates the example interface 600A in which a medical provider 604, such as medical provider 108, 208, 306, 404, and 514. may indicate that the patient refuses a suggested procedure.

The gaps in care page 602, similar to the main page 302 may include member data 308, such as a name, gender, date of birth, and identifier. In other examples, additional or alternative member data 606 may be included, such as age, ethnicity, or the like. The member data 606 may include the same or different data as member data 308 of the main page 302.

The gaps in care page 602 may include one or more potential gaps in care 608 associated with the member. The gap(s) in care 608 may include one or more screenings, procedures, tests, surgeries, consults, or the like that the member should undergo (collectively referred to herein as procedures). In some examples, the service provider may determine the gap(s) in care 608 based on recommended care guidelines (e.g., health maintenance guidelines, recommended health screenings, etc.), such as those published by the center for disease control or other health organization. In some examples, the service provider may determine the gap(s) in care 608 based on the member data.

In various examples, the gap(s) in care 608 may be presented on the gaps in care page 602 based on a determination that the member should undergo a medical screen, procedure, test, surgery, consult, or the like. For example, based on a determination that the member is above a threshold age and has not had a colon cancer screen in the past two years, the service provider may determine to surface a gap in care 608 associated with the colon cancer screening.

In some examples, the gap(s) in care 608 may be presented on the gaps in care page 602 based on a ranking associated therewith. In some examples, if a level of severity or risk associated with not conducting the related screen, procedure, tec. is above a threshold level of severity or risk, the service provider may surface the gap in care 608. In some examples, based on the ranking (e.g., level of severity, risk, etc.), the gap(s) in care 608 may include a label 610 indicating that the gap(s) in care 608 are required to review. In some examples, the label 610 indicating that the gap(s) in care 608 are required to review may be based on a time associated with addressing the one or more gaps in care 608. In such examples, the service provider may determine a time associated with each gap in care 608 and may determine, based at least in part on the time and/or a ranking associated with respective gaps in care 608, whether to indicate a requirement to review.

In other examples, the label 610 may indicate that a review of the gap(s) in care 608 is optional or not applicable. In some examples, the review may be optional based on a determination that the level of severity or risk is below a first threshold. In some examples, the review may be not applicable based on a determination that the service provider was unable to identify a gap in care 608 associated with the member.

In various examples, the gaps in care page 602 may include supporting data 612 regarding the identified gap in care 608. The supporting data 612 may provide a justification for the service provider determination that the member should undergo a procedure associated with the gap in care 608. For example, the supporting data 612 may include member eligibility and a last screening date. In some examples, the supporting data 612 may include an indication that the procedure is pre-authorized for payment (e.g., insurance payment). In some examples, the supporting data 612 may include a cost to the member associated with the procedure.

In the illustrative example, the medical provider 604 may respond to a request for information associated with the gap in care 608, such as if the member has undergone the procedure within the threshold time. In some examples, responsive to receipt of input from the medical provider, the service provider may update the member data. In some examples, responsive to receipt of input from the medical provider, the service provider may surface a first window 614, requesting additional information. In some examples, the additional information may include a request for a date in which the screening was completed, a request for an alternative procedure conducted, a date associated with ineligibility for the procedure associated with the gap in care 608, and the like.

In the illustrative example the additional information requested in the first window 614 may include an indication of whether the member is willing to undergo the procedure associated with the gap in care 608, such as based on a selection that the member should undergo the procedure. Responsive to an indication of selection of an option (or input of additional information) associated with the first window 614, the service provider may update member data and/or cause a second window 616 to surface on the gaps in care page 602, requesting additional information.

In various examples, the second window 616 may request additional information associated with a response indicated in the first window 614. For example, a response provided via the first window 614 may generate an additional inquiry about a status, condition, or procedure related to the member. In some examples, the second window 616 may request information unrelated to a response submitted via the first window 614. For example, the service provider may inquire as to whether the medical provider 604 may perform a task (e.g., provide the member a FIT kit) or whether the medical provider 604 would prefer that the service provider complete the task (e.g., to send the FIT kit to the member).

In the illustrative example, the gaps in care page 602 includes a selectable option 618 to save the information about the gaps in care 608. In some examples, the selectable option 618 may be associated with the gaps in care page 602, as a whole. In such examples, responsive to selection of the selectable option 618, the application may save any data input via the gaps in care page 602.

In various examples, responsive to selecting the selectable option 618 and/or other inputs associated with the gaps in care 608 (e.g., information input via gaps in care page 602, such as via the gap in care 608, the first window 614, and/or the second window 616, etc.) may be sent to the service provider, such as message sent responsive to the input. For example, based on an indication that the medical provider 604 wants the service provider to send the member a FIT kit, a message may be sent to the service provider via the application.

In various examples, the information associated with the gaps in care page 602 may be sent to the service provider based on a submission of the clinical assessment associated therewith. For example, based on an indication that a medical provider 604 has signed and/or submitted a clinical assessment, such as that depicted in FIG. 3, the message may be sent to the service provider indicating the information input via the gaps in care page 602.

FIG. 6B illustrates the example interface 600B in which the medical provider 604 indicates, via the gaps in care page 602, that a referral for the suggested procedure associated with the gap in care 608 will be submitted. In the illustrative example, the additional information requested in the first window 614 may include an indication of whether the member is willing to undergo the procedure associated with the gap in care 608, such as based on a selection that the member should undergo the procedure.

In some examples, responsive to an indication of selection of an option (or input of additional information) associated with the first window 614, such as a willingness to receive a colonoscopy, the service provider may update the member data. In some examples, the responsive to the indication of selection of the option for the member to undergo the procedure (e.g., indication that the medical provider 604 will submit a referral for the procedure) and/or an indication that the selectable option 618 is selected (e.g., saving the data associated with the gaps in care page 602), the service provider may identify one or more locations and/or providers to conduct the procedure. In some examples, the service provider may additionally process data associated with the identified locations and/or providers and may the service provider may cause a referral page with the data to be surfaced via the application.

FIG. 6C illustrates an example interface 600C for submitting a referral 620 via a referral page 622. The referral page 622, similar to the gaps in care page 602 may include member data 606, such as a name, gender, date of birth, and identifier. In other examples, additional or alternative member data 606 may be included, such as age, ethnicity, or the like. In various examples, responsive to receiving an indication that the medical provider intends to submit a referral 620, the service provider may determine whether a referral 620 is duplicative. In such examples, the service provider may determine whether another (e.g., previously submitted) referral for the procedure is outstanding (e.g., procedure not completed). In some examples, based on a determination that the referral 620 is duplicative, the service provider may cause an indication to surface on the referral page 622 indicating that the member is ineligible or that another referral was previously submitted for the procedure.

In various examples, responsive to receiving an indication that the medical provider 604 intends to submit a referral 620 and/or a determination that the referral 620 is not duplicative, the service provider may surface the referral page 622 including one or more options for the referral. The referral page 622 may provide a means by which the medical provider 604 may select one or more options to personalize the referral for the member. In various examples, based on the indication of intention to submit the referral 620 for a procedure associated with the gap in care 608, the service provider may identify one or more locations 624 and/or one or more providers 626 associated with the procedure (e.g., capable of conducting the procedure). In such an example, the options may include at least the one or more locations 624 and/or the one or more providers 626.

As illustrated in FIG. 6C, the identified location(s) 624 and/or providers 626 may be presented on the referral page 622. In some examples, the referral page 622 may additionally include medical provider data associated with the location(s) 624 and/or the provider(s) 626. The medical provider data may include provider location information (e.g., office locations, home address of medical provider, etc.), names and credentials of medical providers associated with a medical office and/or location, clinical visit times (e.g., average time, preferred (target) time, longest visit, shortest visit, etc.), insurance billing history, procedural history, procedural and/or practice specialties, provider quality metric (e.g., based on quality of service (e.g., based on feedback from members, professional organizations, awards earned, etc.), claim submissions (e.g., submitted on time with limited errors, etc.), percentage of patients associated with the service provider, use and management of the service provider application (e.g., clinical assessments, submitting referrals, etc.), ease of scheduling, delays associated with scheduling, etc.).

In various examples, the location(s) 624 and provider(s) 626 may be identified based on an association with a network of the service provider (e.g., an insurance carrier network). In such examples, the location(s) 624 and/or provider(s) 626 may be presented on the referral page 622 based on the association with the network. In some examples, the service provider may additionally surface locations 624 and/or providers 626 that are outside the network. In some examples, the location(s) 624 and provider(s) 626 may be identified based on a certification, specialty, and/or qualification associated with the location(s) 624 and provider(s) 626. In such examples, the service provider may cause relevant location(s) 624 and provider(s) 626 to surface on the referral page 622.

In various examples, the location(s) 624 and provider(s) 626 may be identified based on locations associated therewith and a location associated with the member 628 (e.g., member location 628). In some examples, the location(s) 624 and provider(s) 626 may be identified based on a determination that the locations associated therewith are within a threshold distance 630 of the member location 628. In some examples, the location(s) 624 and provider(s) 626 may be identified based on a determination of one or more closest location(s) 624 and/or provider(s) 626 to the member location 628. For example, a member who lives in a remote area may have to travel a distance (e.g., greater than the threshold 630) to visit a medical professional qualified to conduct the procedure. The service provider may thus provide the member with one or more of the closest locations 624 and/or providers 626. In the illustrative example, the identified location(s) 624 and/or the provider(s) 626 may be depicted on a map 632, such as to provide a visual depiction of the associated locations. In other examples, the identified location(s) 624 and/or the provider(s) 626 may be presented in a list. In some examples, the list may include a ranked list based on location, quality, and/or cost to the member. For example, a procedure conducted at a second location 624(2), Holly Hospital, may be more expensive than the same procedure conducted at a first location 624(1). Accordingly, the second location 624(2) may be presented on the list after (e.g., at a lower position in the list) than the first location 624(1).

In some examples, the service provider may cause a pre-determined number (e.g., 5, 10, etc.) of locations 624 and/or providers 626 to surface on the referral page 622. In some examples, the service provider may cause any locations 624 and/or providers 626 identified to surface, up to a threshold number of locations 624 and/or providers 626 (e.g., no more than 7, 9, etc.). In the illustrative example, the service provider causes four locations 624 and/or providers 626 to surface on the referral page 622, though this is just an example and a greater or lesser number of locations 624 and/or providers 626 are contemplated herein.

In some examples, the locations associated with the location(s) 624 and provider(s) 626 may be determined based on medical provider data. In such examples, the service provider may access the medical provider data to identify the location(s) 624 and provider(s) 626 based on the associated locations being with the threshold distance 630. In various examples, the service provider may determine the member location 628 based on member data, such as a home location (e.g., primary residence, secondary residence, etc.), a work location, or the like. In some examples, a primary or secondary residence may be determined based on a date associated with the referral 620 and/or requested procedure. For example, a member may indicate to the service provider that a primary residence is relevant from March to October and a secondary residence is relevant from November to February. Based on an indication that the referral 620 is submitted on a particular date and/or the service is requested to be scheduled during a particular month, the service provider may identify the location(s) 624 and provider(s) 626 relevant to the primary or secondary residence (e.g., within the threshold distance 630, closest to the member location 628, etc.).

In some examples, the member location 628 may include a current location associated with the member. In some examples, the service provider may determine the current location based on a location associated with the medical provider 604 submitting the referral 620. For example, the service provider may assume that the medical provider 604 accessing the clinical assessment associated with the referral page 622 is co-located with the member (e.g., that the member and the service provider are engaged in a clinical visit). In some examples, the service provider may determine the current location of the member based on a location signal received from a computing device associated with the member. In such examples, a location component (e.g., GPS component, cellular identification component, inertial sensor, Bluetooth beacon, or other component, etc.) of the computing device may determine the current location associated with the member. In some examples, the computing device may send the location signal responsive to a request from the service provider. In some examples, the service provider may identify the location(s) 624 and provider(s) 626 within the threshold distance 630 of the current location of the member.

In various examples, the service provider may identify the location(s) 624 and provider(s) 626 based on a quality metric associated therewith. In various examples, the service provider may determine the quality metric based on the medical provider data. The quality metric may be based on a quality of service (e.g., based on feedback from members, professional organizations, awards earned, etc.), claim submissions (e.g., submitted on time with limited errors, etc.), percentage of patients associated with the service provider, use and management of the service provider application (e.g., clinical assessments, submitting referrals, etc.), ease of scheduling, delays associated with scheduling, and the like. In some examples, the service provider may cause a pre-determined number (e.g., 2, 5, etc.) number of locations 624 and/or providers 626 with a quality metric above a threshold to be surfaced on the referral page 622. In some examples, the service provider may cause each of the locations 624 and/or providers 626 with quality metrics above the threshold to be surfaced. In some examples, the service provider may cause one or more locations 624 and/or providers 626 within the threshold distance 630 and/or closest to the member location 628 and with the quality metric above the threshold to be surfaced on the referral page 622.

In various examples, the quality metric information associated with the location(s) 624 and provider(s) 626 may be presented to the medical provider 604 via the referral page 622. In some examples, the service provider may cause the quality metric information and/or other medical provider data to surface based on an indication that the medical provider 604 hovers over a location associated with the location(s) 624 and/or the provider(s) 626. In some examples, the quality metric information and/or other medical provider data may be presented in the member eligibility section 634.

In various examples, the member eligibility section 634 may additionally or alternatively include information relevant to the referral process, such as to inform the medical provider 604 and/or the member about different options for the procedure, pre-approvals for select location(s) 624 and/or providers 626 certified to conduct the procedure.

In various examples, the service provider may maintain a list of locations and/or providers authorized for pre-approval. In such examples, responsive to receiving an indication of intent to submit a referral 620 and/or a geolocation of the member, the service provider may cause an indication of the pre-approval status to be presented via the referral page 622. For example, the service provider may determine that the Rectal Center (location 624(1) and Dr. Kyle Smith (provider 626(1) are pre-approved for the procedure. The service provider may provide an indication on the map 632 and/or in the member eligibility section 634 of the pre-approval. In various examples, the service provider may provide pre-approved location(s) 624 and/or provider(s) 626 to encourage referrals 620 to the particular location(s) 624 and/or provider(s) 626, such as based on a lower cost option.

In some examples, the pre-approved location(s) 624 and/or provider(s) 626 may be pre-approved based on one or more factors. The factor(s) may include a quality metric, a cost of the procedure corresponding to the location(s) 624 and/or provider(s) 626, or other factors.

In some examples, the member eligibility section 634 may include one or more notes 636 providing additional information about the location(s) 624 and/or provider(s) 626 and/or a justification for why a first location 624 or first provider 626(1) is pre-approved, but a second location 624 or a second provider 626(2) is not. In the illustrative example, the note(s) 636 include an indication that a selection of the second location 624(2) will result in a higher cost for the member than another location and that selection of the second location 624(2) may result in a delay of the procedure, such as based on additional time to receive member approval, processing an approval for the second location 624(2), and the like. Additionally, in the illustrative example, the note(s) 626 include a warning to the medical provider that the second provider 626(2) is out of network and may result in significant expense to the member. In some examples, the out of network provider may be selected and the member may accept the increased cost associated therewith. For example, a member may have one location 624 or provider 626 within 200 miles of a member location 628 to have the procedure, and the one location 624 or provider 626 may be out of network and/or may include a greater expense to the member than another location 624 or provider 626 a greater distance away. The service provider may provide a means by which the member may accept the additional cost, such as by sending a message to a member computing device, such as member computing device 110, requesting acceptance of the additional cost.

In various examples, the referral page 622 may provide a means by which the medical provider 604 may select a particular location 624 or provider 626 for the referral 620. As an illustrative example, the medical provider 604 selects Dr. Kyle Smith for the referral 620. The medical provider 604 may submit the referral 620 by selecting the submit referral selectable option 638. In various examples, responsive to an indication of selection of the submit referral selectable option 638, the application may cause the referral 620 to be sent to the service provider.

FIG. 7 illustrates another example interface 700 associated with submitting a referral 702 for treatment, such as at a time associated with or independent of a clinical visit. In various examples, the medical provider 704, such as medical provider 108, 208, 306, 404, 514, and 604, may submit the referral 702 via an application associated with a service provider, such as service provider 104. In such examples, the medical provider 704 may access the application and submit the referral 702.

In some examples, the medical provider 704 may submit the referral 702 for a procedure via a web site 706 associated with the service provider. As discussed above, the medical provider 704 may submit the referral 702 based on an indication of a gap in care associated with a member 708, such as gap in care 608. In some examples, the medical provider 704 may submit the referral 702 responsive to an indication that the member 708 is due for the procedure. In such examples, the service provider may send the medical provider 704 the indication, such as via a push notification, a text message, electronic mail message, a telephone message, or other means of communicating information between the service provider and the medical provider 704.

The referral may include boxes to input member data 710. In the illustrative example, the boxes to input member data 710 include a box for a name, date of birth and identifier. In other examples, the boxes to input member data 710 may include additional or alternative information, such as an age, race, ethnicity, or any other information used to identify the member 708.

In some examples, the interface 700 may include a procedure entry box 712. The procedure entry box 712 may include a list of one or more procedures eligible for the referral 702. In the illustrative example, the procedure entry box 712 may include a drop-down menu 714 including the list of procedure(s). In some examples, particular procedures in the list of procedures may include an indication 716 that the procedure may be eligible for automatic approval. In some examples, the service provider may cause the indication 716 to be associated with the procedures based in part on the member, such as based on an insurance policy associated therewith, an age, a gender, a race, an ethnicity, or the like. For example, the service provider may determine that based on demographic information associated with the member, that a particular disease may be more common for the member. Based at least in part on the demographic information, the service provider may determine that the procedure may be pre-approved. In various examples, the service provider may utilize one or more machine learning models to determine whether a procedure may be automatically approved. In some examples, the machine learning models may be trained to output procedures eligible for automatic approval based on member data 710, statistics associated with the procedure, and he like. In some examples, the automatic approval may encourage members to undergo procedures as a preventative and/or precautionary measures, such as to maintain a good state of health.

In the illustrative example, the medical provider 704 selects endoscopy from the list in the procedure entry box 712. In various examples, responsive to a selection of a procedure in the procedure entry box 712, the service provider may cause a location entry box 718 and/or a provider entry box 720 to surface on the interface 700. The location entry box 718 may be associated with a list of locations 722 at which the member 708 may undergo the procedure and the provider entry box 720 may include a list of providers 724 eligible to perform the procedure. The service provider may identify the location(s) 722 and/or providers 724 included in the respective entry boxes 718 and 720 utilizing the techniques described above with regard to FIG. 6C.

In some examples, the service provider may cause one or both of the location entry box 718 or the provider entry box 720 to be presented on the interface 700. In such examples, the locations 722 and the provider(s) 724 may be associated with one another or independent of one another. For example, a first interface 700 may include a location entry box 718, enabling the medical provider 704 to refer the member 708 to a location 722 and a second interface 700 may include a provider entry box 720, enabling the medical provider 704 to refer the member 708 to a particular medical provider 724. In some examples, responsive to receiving an input regarding a provider 724 or a location 722, the service provider may cause the other of the location entry box 718 or the provider entry box 720 to surface on the interface. For example, responsive to the medical provider selecting the “INTERNAL DIGESTION” as a location 722, the service provider may surface the provider entry box 720 including the list of providers 724 associated with the location.

In various examples the locations(s) 722 and/or the provider(s) 724 may include cost indications 726. The cost indications 726 may include a potential cost to the customer corresponding to a procedure undergone at the associated location 722 and/or performed by the associated provider 724. In some examples, the cost indication 726 may encourage the medical provider 704 to refer the member 708 to a less expensive location 722 and/or 724. In the illustrative example, the list of locations 722 may include the cost indication 726, however this is not intended to be limiting (e.g., one or both of the locations 722 and the providers 724 may include the cost indication 726).

As illustrated in FIG. 7, the providers 724 may include an indication of quality 728. In other examples, one or more of the providers 724 and/or one or more of the locations 722 may include the indication of quality. The indication of quality 728 may be based on the quality metric described above. The service provider may determine the quality metric based on quality of service (e.g., based on feedback from members, professional organizations, awards earned, etc.), claim submissions (e.g., submitted on time with limited errors, etc.), percentage of patients associated with the service provider, use and management of the service provider application (e.g., clinical assessments, submitting referrals, etc.), ease of scheduling, delays associated with scheduling, and the like.

In some examples, the indication of quality 728 may be associated with a location 722 and/or a provider 724 based on a determination that the quality metric is above a threshold quality level. In the illustrative example, the indication of quality 728 may provide an indication of a level of quality, such as based on a number of stars associated with the indication of quality 728. In various examples, an automatic approval of the procedure, such as that described above, may be determined based at least in part on a selected location 722 and/or a selected provider 724 and/or an indication of quality 728 associated therewith. In some examples, the machine learning models discussed above may be trained to output the automatic approval for the procedure based in part on the selected location 722, the selected provider 724, and/or the indication of quality 728 associated with the selected location 722 and/or the selected provider 724.

In various examples, the interface 700 may include additional notes section 730. In such examples, the additional notes section 730 may include additional information associated with the referral 702, the procedure, a selected location 722 and/or a selected provider 724. In the illustrative example, the additional notes section 730 may include an indication that an option may be available to lower a cost to the member 708 associated with the referral 702. Additionally, in illustrative example, the additional notes section 730 may include a selectable option 732 for accessing additional information about the note. In some examples, the selectable option 732 may include a hyperlink to a different website, such as that associated with the selected location 722 and/or the selected provider 724. In some examples, responsive to selection of the selectable option 732, the service provider may cause a window 734 to surface on the interface 700. In some example, the window 734 may include the additional information.

In some examples, the referral 702 may be associated with a referral for a particular piece of equipment. For example, the referral 702 may be associated with a speech pathology device. In some examples, the service provider may be configured to identify an alternative piece of equipment that may perform the same or a similar function as the particular piece of equipment, but at a greatly reduced cost to the member 708. In the illustrative example, the additional notes section 730 includes an indication that different equipment may potentially be available for the member 708. In such an example, responsive to selecting the selectable option 732, the medical provider 704 may access information about the alternative piece of equipment, such as via the window 734. In some examples, the window 734 may include a means by which the medical provider may select the alternative equipment.

The medical provider 704 may submit the referral 702 by selecting the submit referral selectable option 736. In various examples, responsive to an indication of selection of the submit referral selectable option 736, the service provider may receive the referral 702

FIG. 8 illustrates an example interface 800 corresponding to a clinical recommendations page 802 associated with a clinical assessment, such as clinical assessment 118 and 304. The clinical recommendations page 802, similar to the main page 302 may include member data 804, such as a name, gender, date of birth, and identifier. In other examples, additional or alternative member data 804 may be included, such as age, ethnicity, or the like. The member data 804 may include the same or different data as member data 308 of the main page 302.

The clinical recommendations page 802 may include one or more clinical recommendations 806 associated with the member. As discussed above, the service provider may determine the one or more clinical recommendations 806 based at least in part on member data. In some examples, the clinical recommendations 806 may be determined based at least in part on one or more machine learning models. In such examples, the machine learning models may be trained to output one or more clinical recommendations 806 based on training data including member data, (e.g., known diagnoses, age, laboratory results (e.g., blood sugar, cholesterol, etc.)).

In various examples, the clinical recommendation 806 may include a recommendation for the medical provider 808, such as medical provider 108, 208, 306, 404, 514, 604, and 704, to prescribe a particular medication, procedure, screen, test, or the like for the member. In the illustrative example, the clinical recommendation 806 includes a recommendation for the medical provider 808 to prescribe an injectable therapy.

In various examples, the interface 800 may include supporting evidence 810, providing a justification and/or support for the clinical recommendation 806. In the illustrative example, the supporting evidence 810 includes a relevant member history, including a diagnosis, medications, lab results, and clinical guidelines 812 supporting the clinical recommendation 806. Additionally, in the illustrative example, the supporting evidence may include a link 814 to access additional information about the clinical guidelines 812. The link 814 may include a hyperlink to another website (e.g., relevant guidelines on the other website) and/or may launch a window on the interface 700, such as for a quick review of the clinical guidelines 812. In other examples, the supporting evidence 810 may include additional or alternative information to support the clinical recommendation and/or provide a justification for why the service provider has caused it to surface via the application associated with a clinical assessment.

In some examples, the clinical recommendation page 802 may include one or more clinical recommendations 806 determined by the service provider. In some examples, the one or more clinical recommendations 806 may be presented on the clinical recommendation page 802 based on a ranking (e.g., level of severity, etc.), such as that described above. In the illustrative example, the clinical recommendation page 802 includes a single clinical recommendation 806 with a label 816 indicating a requirement to address the clinical recommendation 806. In other examples, the clinical recommendation page 802 may not include any clinical recommendation 806, such as if the service provider determines that no clinical recommendations 806 are relevant to the clinical assessment. In such examples, the label 816 may indicate that the requirement to address a potential diagnosis is not applicable.

In various examples, the clinical recommendation 806 may have associated therewith the requirement to address (e.g., label 816) based on a ranking associated with the clinical recommendation 806. As discussed above, the ranking may include a level of severity associated with the clinical recommendation 806 (e.g., an importance of the clinical recommendation 806 to the member). In some examples, the clinical recommendation 806 may be included on the clinical recommendation page 802 based on the level of severity being above a threshold level of severity. In some examples, based on a determination that the level of severity is below the threshold level of severity, the label 816 may indicate that a review of the clinical recommendation 806 is optional.

In some examples, a determination that a clinical recommendation 806 is required to review or optional to review may be based on an estimated time to address the clinical recommendation 806 and/or a total time associated with the potential diagnoses, the medications, and/or the gaps in care described above. In some examples, the determination may be based on a ranking of the clinical recommendation 806 related to rankings associated to the potential diagnoses, the medications, and/or the gaps in care. For example, based on a determination that a potential diagnosis is extremely important to confirm during a clinical visit and/or that the potential diagnosis has an estimated review time equal to or greater than the total time for the clinical visit, the service provider may determine to make the clinical recommendation 806 optional.

In various examples, the clinical recommendation 806 may include one or more selectable options 820 for submitting input associated with the clinical recommendation 806. In such examples, responsive to selecting one of the one or more selectable options 820, the service provider may receive an indication of selection and/or may initiate an approval process associated therewith. In the illustrative example, the selectable options 820 include potential drugs that could be prescribed to address the clinical recommendation 806. In some examples, the selectable options 820 may include a cost indication 822, indicating a cost to the member of a respective option. In various examples, the cost indication 822 may encourage the medical provider 808 to select a less expensive option to prescribe for the member.

In some examples, the clinical recommendation 806 may include a response 824 to the clinical recommendation 806. In some examples, the response 824 may include a means by which the medical provider 808 may indicate whether the clinical recommendation 806 was helpful to providing medical care. In the illustrative example, the response 824 includes an indication of whether or not the medical provider 808 prescribed the suggested medication associated with the clinical recommendation 806. In some examples, the interface 700 may additionally include a window, such as window 428 or 520, requesting a justification for why the clinical recommendation 806 was not followed. In such examples, the service provider may use the information provided in the justification to improve a recommendation component, such as recommendation component 122 configured to determine clinical recommendations 806. In some examples, the justification may be used to train the machine learning model to output more accurate and/or relevant clinical recommendations.

In the illustrative example, the clinical recommendations page 802 includes a selectable option 826 to save the information about the clinical recommendation 806. In some examples, the selectable option 826 may be associated with the clinical recommendations page 802, as a whole. In such examples, responsive to selection of the selectable option 826, the application may save any data input via the clinical recommendations page 802.

In various examples, responsive to selecting the selectable option 826 and/or other inputs associated with the clinical recommendations 806 (e.g., information input via the clinical recommendations page 802, such as via the selectable options 820, the response 824, etc.) may be sent to the service provider, such as message sent responsive to the input.

In various examples, the information associated with the selectable options 820 may be sent to the service provider based on a submission of the clinical assessment associated therewith. For example, based on an indication that a medical provider 808 has signed and/or submitted a clinical assessment, such as that depicted in FIG. 3, the message may be sent to the service provider indicating the information input via the clinical recommendations page 802.

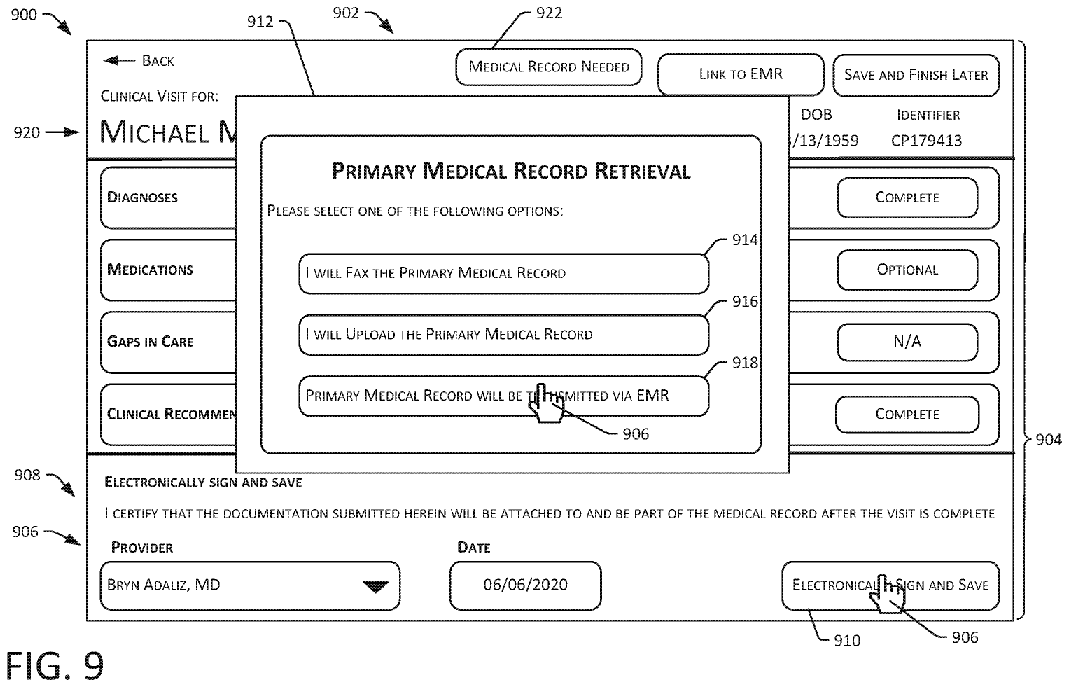

FIG. 9 illustrates an example interface 900 associated with a main page 902 through which a clinical assessment 904, such as clinical assessment 118, 202, and 304, may be signed and submitted by a medical provider 906, and a patient medical record may be verified. As discussed above with regard to FIG. 3, the main page 902 may include a signature section 908, such as signature section 324. The signature section 908 may provide a means by which the medical provider 906 may electronically sign the clinical assessment 904 and save data associated therewith. In the illustrative example, the medical provider 906 may electronically sign the clinical assessment 904 by selecting a selectable option 910. As illustrated in FIG. 9, the selectable option 910 may be labeled “ELECTRONICALLY SIGN AND SAVE,” though this is for illustrative purposes and is not intended to be limiting. For example, the selectable option 910 may include a label “SIGN AND SUBMIT,” or any other label to indicate a signature, storing action, and/or submission to the service provider.

In some examples, responsive to receiving an indication of selection of the selectable option 910, a signed clinical assessment 904 (e.g., supplemental medical record) may be submitted to the service provider. In some examples, responsive to receiving an indication of selection of the selectable option 910, the service provider may cause a medical record retrieval window 912 to be presented via the main page 902. As illustrated, the medical record retrieval window 912 may provide a means by which the medical provider 906 may indicate a source through which they will share a patient's primary medical record. In other examples, an office manager or other staff member associated with the medical provider may surface the medical record retrieval window 912, to provide the information associated therewith to the service provider. For example, the medical record retrieval window 912 may be surfaced via a first instance of an application on a first computing device associated with a medical provider 906 office (e.g., office assistant computing device) or a second instance of an application on a second computing device associated with the medical provider 906 (e.g., medical provider device).

In some examples, the medical record retrieval window 912 may include a first option 914 to fax the primary medical record, a second option 916 to upload the primary medical record (e.g., upload a PDF or other file format of the medical record), and a third option 918 to transmit the primary medical record via an electronic medical record. Though other methods of transmitting data associated with a primary medical record are contemplated herein. Responsive to receiving an indication of selection of the first option 914, the second option 916, or the third option 918, the service provider may store the data and monitor the means of transmission for the primary medical record. Responsive to receiving the primary medical record via the means, the service provider may match the primary medical record with the supplemental record (e.g., clinical assessment 904). For example, responsive to receiving an indication of selection of the second option 916, the service provider may monitor uploaded files to identify a particular file corresponding to a primary medical record that is associated with a particular clinical assessment 904. For another example, responsive to receiving an indication of selection of the first option 914, the service provider may monitor and/or analyze faxed documents to identify a particular file corresponding to a primary medical record that is associated with a particular clinical assessment 904.

In the illustrative example, the medical provider 906 (or associate thereof) may select the third option 918 to transmit the primary medical record via an electronic medical record. In some examples, the electronic medical record may be transmitted automatically based on an integration between the service provider and an electronic medical record system associated with the medical provider 906. In such examples, the electronic medical record system and the service provider may communicate and transmit data back and forth based on a determination that a clinical assessment 904 is complete (e.g., signed and saved), scheduled, and/or due to be scheduled. In some examples, primary medical record data associated with a particular member 920 (e.g., patient) may be automatically transmitted to the service provider periodically (e.g., every six months, annually, etc.). In such examples, the service provider may maintain up to date information associated with the particular member 920, regardless of whether clinical assessments 904 occur.

In various examples, the service provider may receive the primary medical record associated with a member 904 and may match the primary medical record and the clinical assessment 904 data (e.g., supplemental medical record). In various examples, the service provider may update member data and/or process an insurance claim associated with the clinical visit. In some examples, the service provider may additionally process an approval for a referral submitted with the clinical assessment 904, such as via the gaps in care section discussed above with regard to FIGS. 6B and 6C.

In various examples, the service provider may determine that a primary medical record associated with a clinical assessment 904 has not yet been received. In some examples, the service provider may determine that the primary medical record has not been received within a threshold amount of time of the clinical assessment 904 (e.g., an appointment time, a completion time, etc.) and/or submission thereof, and may cause an alert 922 to be presented via the main page 902. In some examples, the alert 922 may be presented based on a determination that the clinical assessment was not matched to a primary medical record within a second threshold amount of time (e.g., within 4 hours, 8 hours, 24 hours, etc.).

In various examples, the alert 922 may provide an indication to the medical provider 906 and/or an associate thereof that the primary medical record has not been received and/or matched to a clinical assessment 904. In some examples, the alert 922 may prompt the medical provider 906 and/or associate thereof to upload or otherwise send the medical record to the service provider. In some examples, the alert 922 may prompt the medical provider 906 and/or associate thereof to resolve an issue that prevented the clinical assessment 904 to be matched to a primary medical record. For example, if a clinical assessment 904 includes an incorrect date of service or there is a typo associated with patient 920 data, the error may prevent the clinical assessment 904 from being matched to the medical record. In examples in which a matching failure has occurred, the alert 922 may include an indication for the medical provider 906 and/or associate thereof to check the clinical assessment 904 for errors.

In various examples, the service provider may monitor the number of successfully matched (e.g., paired) primary medical records and supplemental medical records (e.g., clinical assessments 904). In some examples, the service provider may generate reports associated with a number and/or percentage of successfully paired medical records over time. In some examples, the number and/or percentage of successfully paired medical records may be used by the service provider to drive improvements in behavior over time. For example, different alerts may be used and information provided when a match between a primary and supplemental medical record does not occur. The success rates associated with subsequent matchings may be monitored and used to inform decisions of the types of alerts and information provided that is most successful to the end user (e.g., medical provider 906).

### Example Computing Architecture

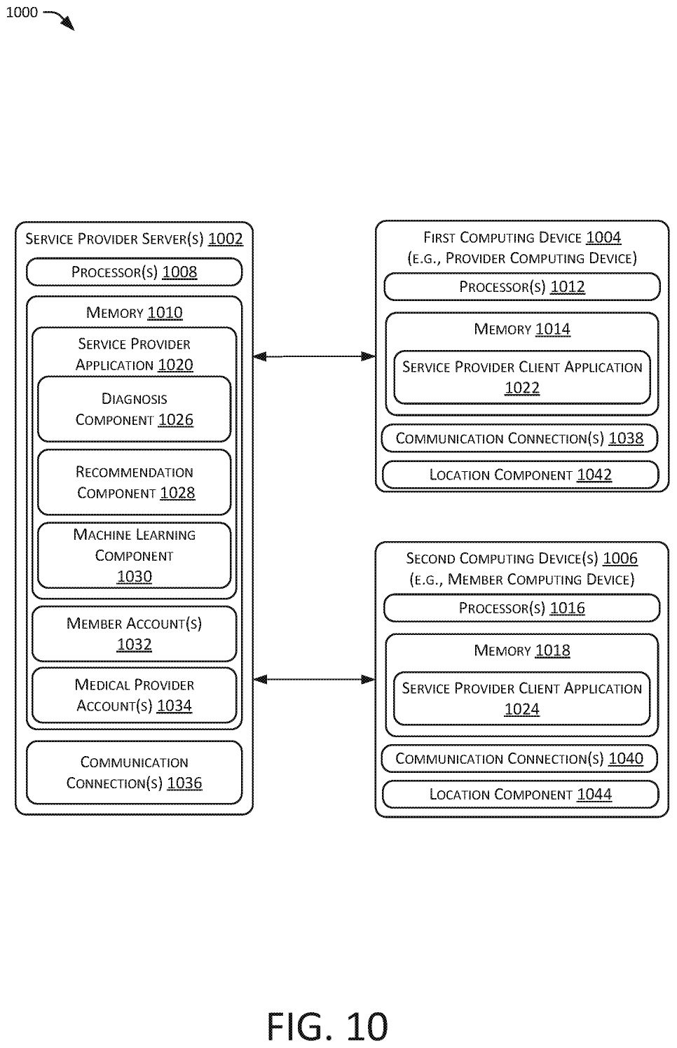

FIG. 10 illustrates a block diagram illustrating an example system 1000 of computing devices usable to implement example techniques described herein. For example, FIG. 10 illustrates example computing devices including service provider server(s) 1002, one or more first computing devices 1004, and one or more second computing devices 1006, that interact over a network, such as network 114 in FIG. 1. By way of example and not limitation, the service provider server(s) 1002 may be representative of servers used to implement the system 100, the first computing device(s) 1004 may be representative of the medical provider computing device 106 associated with the medical provider 108, and the second computing device(s) 1006 may be representative of the member computing device 110 associated with the member 112.

The service provider server(s) 1002 may comprise one or more individual servers or other computing devices that may be physically located in a single central location or may be distributed at multiple different locations. The service provider server(s) 1002 may be hosted privately by an entity administering all or part of the communications network (e.g., a utility company, a governmental body, distributor, a retailer, manufacturer, etc.), or may be hosted in a cloud environment, or a combination of privately hosted and cloud hosted services.

Each of the computing devices described herein may include one or more processors and/or memory. Specifically, in the illustrated example, service provider server(s) 1002 include one or more processors 1008 and memory 1010, first computing device(s) 1004 includes one or more processors 1012 and memory 1014, and second computing device(s) 1006 includes one or more processors 1016 and memory 1018. By way of example and not limitation, the processor(s) may comprise one or more Central Processing Units (CPUs), Graphics Processing Units (GPUs), or any other device or portion of a device that processes electronic data to transform that electronic data into other electronic data that may be stored in registers and/or memory. In some examples, integrated circuits (e.g., ASICs, etc.), gate arrays (e.g., FPGAs, etc.), and other hardware devices may also be considered processors in so far as they are configured to implement encoded instructions.

The memory may comprise one or more non-transitory computer-readable media and may store an operating system and one or more software applications, instructions, programs, and/or data to implement the methods described herein and the functions attributed to the various systems. In various implementations, the memory may be implemented using any suitable memory technology, such as static random-access memory (SRAM), synchronous dynamic RAM (SDRAM), nonvolatile/Flash-type memory, or any other type of memory capable of storing information. The architectures, systems, and individual elements described herein may include many other logical, programmatic, and physical components, of which those shown in the accompanying figures are merely examples that are related to the discussion herein.

As shown in FIG. 10, service provider server(s) 1002 include a service provider application 1020, first computing device(s) 1004 includes service provider client application 1022, and second computing device(s) 1006 includes service provider client application 1024 that enables interaction of content among the computing devices via the service provider server(s) 1002. For example, content (e.g., member data, medical provider data, scheduling data, referral data, insurance data, etc.) can be shared among users associated with service provider accounts (e.g., member accounts, medical provider accounts, etc.) of an insurance provider network provided by the service provider system and may include sharing content in accordance with an account of a user that is restricted, such as based on health information privacy rules and/or regulations. In some examples, the service provider client application enables interfaces to access content, to view content, and to generate content as those described with reference to FIGS. 2A-9, for example. In particular examples, service provider server(s) 1002 send instructions to present, transmit, and receive content as discussed with reference to FIG. 2A-FIG. 9.

FIG. 10 further illustrates service provider server(s) 1002 as including diagnosis component 1026, recommendation component 1028, and machine learning component 1030 to enable content such as potential diagnoses, medications, gaps in care, clinical recommendations, referrals, and the like, to be shared among the computing devices. In various examples, the diagnosis component 1026 may be configured to generate on or more potential diagnoses associated with a member. In some examples, the potential diagnoses may be determined based on member data, such as that stored in one or more member accounts 1032. In various examples, the potential diagnoses may be determined utilizing machine learning techniques. In such examples, the diagnosis component 1026 may include one or more machine learning models configured to output the potential diagnoses and/or a probability associated therewith based on member data, statistics, and/or other relevant information. In various examples, the diagnosis component may be configured to determine a time associated with each potential diagnosis. In such examples, the time may represent an estimated time for a medical provider to review the potential diagnosis, supporting evidence, and the like with the member, such as to confirm the condition or determine that the member does not have the condition. In various examples, the diagnosis component 1026 may be able to determine a level of severity and/or probability associate with a potential diagnosis and/or rank the potential diagnoses based on the ranking.

In various examples, the recommendation component 1028 may be configured to determine medication information to include in the clinical assessment, as described herein. In some examples, the recommendation component 1028 may be configured to determine a time associated with a review of each medication and/or a ranking associated with one or more medications, such as that described above.

In some examples, the recommendation component 1028 may be configured to determine one or more gaps in care associated with the member, such as based on member data utilizing the techniques described above. In some examples, the recommendation component 1028 may be configured to determine a time associated with a review of the gaps in care and/or a ranking associated with the one or more gaps in care.

In some examples, recommendation component 1028 may be configured to determine one or more clinical recommendations associated with a member, such as based on member data utilizing the techniques described above. In some examples, the recommendation component 1028 may be configured to determine a time associated with a review of the clinical recommendations and/or a ranking associated with the one or more clinical recommendations.

In some examples, recommendation component 1028 may be configured to determine one or more locations and/or providers to surface to a medical provider such as during a referral process, utilizing the techniques described above. In some examples, the locations and/or providers may be determined based on medical provider data, such as that stored in one or more medical provider accounts 1034.

In some examples, the machine learning component 1030 may be configured to train the one or more machine learning models associated with the diagnosis component 1026 and/or the recommendation component 1028, such as based on applicable training data.

In various examples, the service provider application 1020 may be configured to determine one or more of the diagnoses, the medications, the gaps in care, and/or the clinical recommendations to include in a clinical assessment. In some examples, the service provider application may determine whether to require a review of the one or more of the diagnoses, the medications, the gaps in care, and/or the clinical recommendations, such as described above. In some examples, the determination to include the diagnoses, the medications, the gaps in care, and/or the clinical recommendations and or the determination regarding the requirement to review may be based in part on rankings associated with each of the diagnoses, medications, gaps in care, and/or clinical recommendations. In some examples, the service provider application 1020 may be configured to determine an overall ranking of the diagnoses, the medications, the gaps in care, and/or the clinical recommendations. In some examples, the overall ranking may be used to determine which of the diagnoses, the medications, the gaps in care, and/or the clinical recommendations to include in the clinical assessment and/or require for review (or make optional, not applicable, etc.).

In some examples, the service provider application 1020 may determine one or more of the diagnoses, the medications, the gaps in care, and/or the clinical recommendations to include in a clinical assessment based on medical provider data associated with the corresponding medical provider, such as that stored in a medical provider account 1034. The medical provider data may include one or more preferences the medical provider may have regarding the service provider client application 1022. The preferences may include a preference as to whether to include one or more potential diagnoses, medications, gaps in care, and/or clinical recommendations in a clinical assessment, a total time allocated for clinical assessments, a format associated with the clinical assessment (e.g., how the data is displayed), and the like. In various examples, the service provider application 1020 may cause data to be included in the clinical assessment based on the preferences.

As shown in FIG. 10, service provider server(s) 1002 may include communications connection(s) 1036, first computing device(s) 1004 may include communications connection(s) 1038, and second computing device(s) 1006 may include communications connection(s) 1040 that enable communication between at least the service provider server(s) 1002 and one or more of the first computing device(s) 1004, and the second computing device(s) 1006.

The communication connection(s) 1036, 1038, and/or 1040 may include physical and/or logical interfaces for connecting service provider server(s) 1002, first computing device(s) 1004, and/or second computing device(s) 1006 to another computing device or a network, such as network(s) 114. For example, the communications connection(s) 1036, 1038, and/or 1040 may enable Wi-Fi-based communication such as via frequencies defined by the IEEE 802.11 standards, short range wireless frequencies such as Bluetooth®, cellular communication (e.g., 2G, 2G, 4G, 4G LTE, 5G, etc.) or any suitable wired or wireless communications protocol that enables the respective computing device to interface with the other computing device(s).

As shown in FIG. 10, the first computing device(s) 1004 may include a location component(s) 1042, and second computing device(s) 1006 may include location component(s) 1044 that enable the respective computing device(s) 1004 and 1006 to determine a location associated therewith. The location component(s) 1042 and/or 1044 may include one or more of a GPS component, cellular identification component, inertial sensor, Bluetooth beacon, or other component for determining a location of the respective computing device 1004 or 1006. In some examples, the service provider server(s) 1002 may send a request for a current location to the first computing device 1004 or the second computing device 1006, such as during a referral submission process. The location component 1042 or 1044 may determine the current location and cause the respective computing device 1004 or 1006 to send the current location to the service provider server(s) 1002.

While FIG. 10 is provided as an example system 1000 that can be used to implement techniques described herein, the techniques described and claimed are not limited to being performed by the system 1000, nor is the system 1000 limited to performing the techniques described herein.

### Example Methods

FIGS. 10-15 illustrate example processes in accordance with embodiments of the disclosure. These processes are illustrated as logical flow graphs, each operation of which represents a sequence of operations that may be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be combined in any order and/or in parallel to implement the processes.

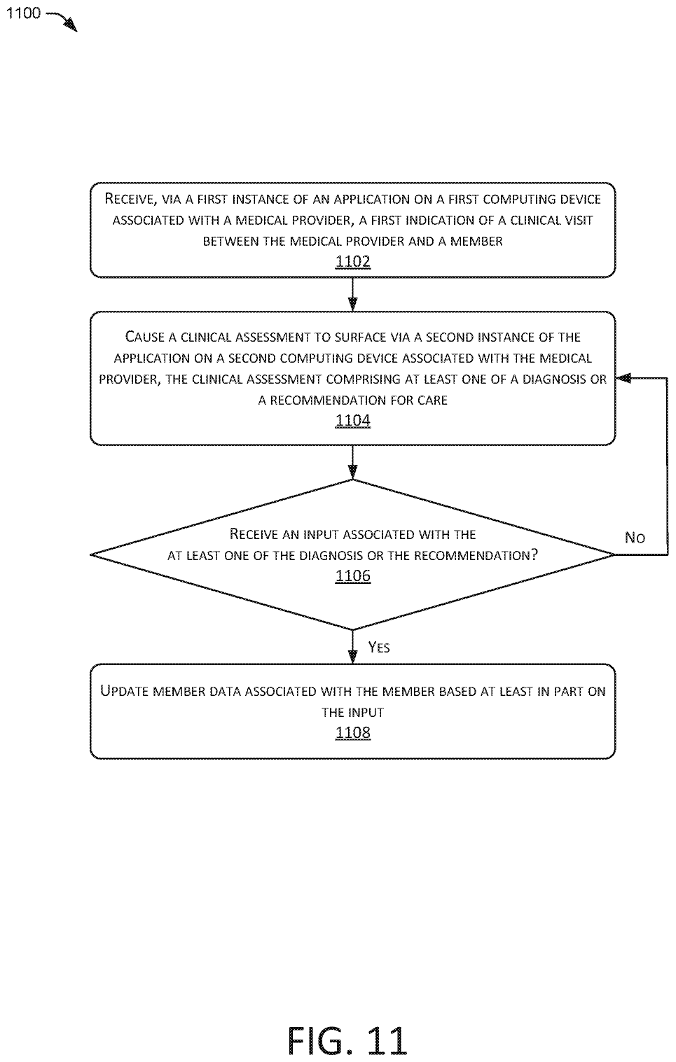

FIG. 11 illustrates an example processes 1100 for surfacing a clinical assessment tool and updating a member record based on input received via the clinical assessment tool, utilizing the techniques described herein. In some instances, some or all of process 1100 may be performed by one or more components in the systems 100 or 1000. By way of example and not limitation, the service provider computing device (e.g., service provider) referred to in process 1100 may be representative of a computing device associated with the service provider 104 or service provider server(s) 1002, the medical provider computing device (e.g., member device) referred to in process 1100 may be representative of the medical provider computing device(s) 106 and/or first computing device(s) 1004 and the member computing device referred to in process 1100 may be representative of the member computing device(s) 110 and/or the second computing device(s) 1006. However, the process 1100 is not limited to being performed by the system 100 or 1000.

At operation 1102, the process 1100 may include receiving, via a first instance of an application on a first computing device associated with a medical provider, a first indication of a clinical visit between the medical provider and a member. In various examples, the first indication may include a request for the service provider to generate a clinical assessment, such as that described above with regard to FIG. 2B. In some examples, the request may include member data, such as that required to identify the member (e.g., name, date of birth, identifier, etc.), a date of service (e.g., date associated with the clinical visit), and the like. Responsive to receiving the first indication of the clinical visit, the service provider may generate a clinical assessment comprising one or more of potential diagnoses, medications, gaps in care, and/or clinical recommendations associated with the member. The clinical assessment may provide the medical provider with relevant information associated with the member, such as to assist the medical provider in maximizing effectiveness and efficiency associated with the clinical visit (e.g., maximize care, minimize time.

At operation 1104, the process 1100 may include causing a clinical assessment to surface via a second instance of the application on a second computing device associated with the medical provider, the clinical assessment comprising at least one of a diagnosis or a recommendation for care. The first instance and the second instance of the application may be the same or different instances of the application and the first and the second computing devices associated with the medical provider may be the same or different computing devices. For example, a medical provider may send the first indication and access the clinical assessment via a single device or different devices.

At operation 1106, the process 1100 may include determining whether input associated with the at least one of the diagnosis or the recommendation is received. The input may be received via one or more of the interfaces 300, 400A, 400B, 500, 600A, 600B, 600C, 700, 800 described in FIGS. 3-8.

Based on a determination that the at least one of the diagnosis or the recommendation is received (“Yes” at operation 1106), the process 1100 may include, at operation 1108, updating member data associated with the member based at least in part on the input. In some examples, the service provider may update a member account associated with the member, such as member account(s) 1032.

Based on a determination that neither one the diagnosis or the recommendation is received (“No” at operation 1106), the process 1100 may include, causing the clinical assessment to surface via the second instance of the application on the second computing device, such as that described with respect to operation 1104.

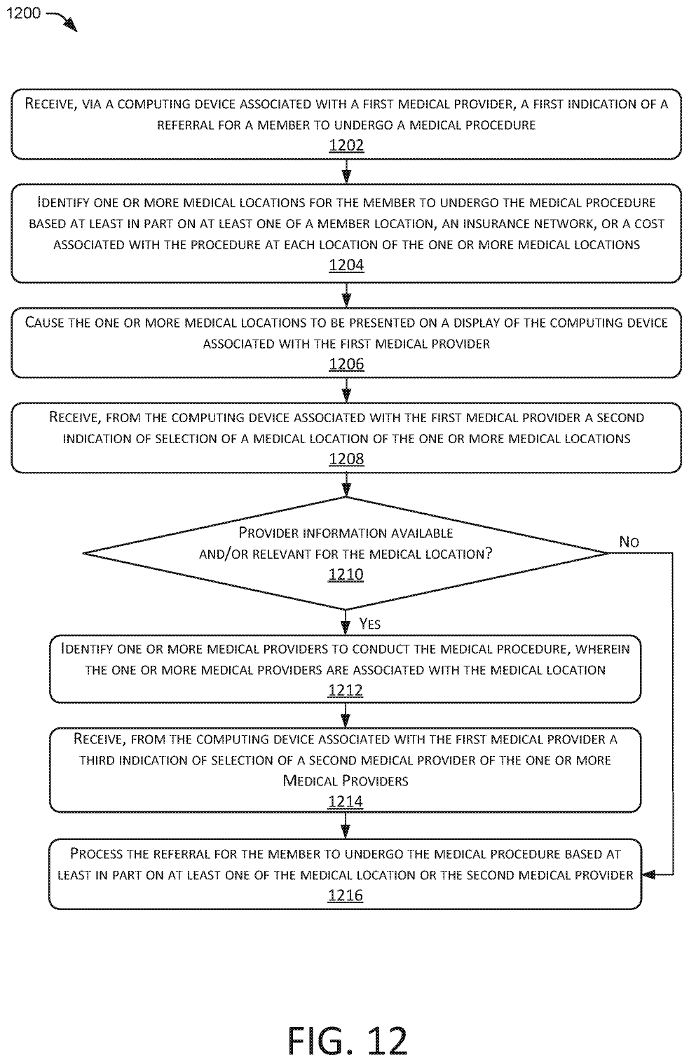

FIG. 12 illustrates an example process for processing a referral submitted by a medical provider, utilizing the techniques described herein. In some instances, some or all of process 1200 may be performed by one or more components in the systems 100 or 1000. By way of example and not limitation, the service provider computing device (e.g., service provider) referred to in process 1200 may be representative of a computing device associated with the service provider 104 or service provider server(s) 1002, the medical provider computing device (e.g., member device) referred to in process 1200 may be representative of the medical provider computing device(s) 106 and/or first computing device(s) 1004 and the member computing device referred to in process 1200 may be representative of the member computing device(s) 110 and/or the second computing device(s) 1006. However, the process 1200 is not limited to being performed by the system 100 or 1000.

At operation 1202, the process 1200 may include receiving, via a computing device associated with a first medical provider, a first indication of a referral for a member to undergo a medical procedure. In some examples, the first indication of the referral may include an input provided via the window 614 of FIG. 6B, indicating that the medical provider would refer the member for the associated procedure (e.g., colonoscopy). In some examples, the first indication of the referral may include a selection of the selectable option 618 of FIG. 6B to submit a referral. In some examples, a first indication of the referral may include an indication that the medical provider has input member identifying information and/or a procedure via a website associated with a referral process, such as website 706 of FIG. 7.

At operation 1204, the process 1200 may include identifying one or more medical locations for the member to undergo the medical procedure based at least in part on at least one of a member location, an insurance network, or a cost associated with the procedure at least location of the one or more medical locations. The medical locations may include locations 624 and/or 722 of FIGS. 6C and 7, respectively. As discussed above, the service provider may be configured to determine the member location based on a location associated with a member computing device, a member location associated with member data, and/or a location associated with the computing device associated with the first medical provider.

In various examples, the medical locations may be identified based on being within a threshold distance of the member location. In some examples, the medical locations may be identified based on being one or more of the closest locations to the member location. In some examples, a number of medical location(s) identified may include a pre-determined number of medical locations within the threshold distance and/or closest to the member location. Additionally or in the alternative, the medical location(s) may be identified based on a capacity of the medical location(s) to complete the medical procedure.

At operation 1206, the process 1200 may include causing the one or more medical locations to be presented on a display of the computing device associated with the first medical provider. The medical location(s) may be presented via an interface, such as interface 600C of FIG. 6B or interface 700 of FIG. 7.

At operation 1208, the process 1200 may include receiving, from the computing device associate with the first medical provider, a second indication of selection of a medical location of the one or more medical locations.

At operation 1210, the process 1200 may include determining whether provider information is available and/or relevant for the medical location. The provider information may be relevant based on a determination of a cost associated with the different providers at the location, a quality metric associated with a provider, an auto-approval status associated with a provider, or other information that may inform a referral and/or scheduling determination.

Based on a determination that the provider information is not available and/or is not relevant for the medical location (“No” at operation 1210), the process 1200 may include, at operation 1216, processing the referral for the member to undergo the medical procedure at the location.

Based on a determination that the provider information is available and/or is relevant for the medical location (“Yes” at operation 1210), the process 1200 may include, at operation 1212, identifying one or more medical providers to conduct the medical procedure, the one or more medical providers being associated with the medical location. In some examples, the medical provider(s) may be identified based on a qualification, certification, specialty, and the like associated therewith, such as that determined based on medical provider data stored in a medical provider account (e.g., medical provider account 1034). In some examples, the medical provider(s) may be identified based on a network associated with the service provider, a quality metric associated therewith, a cost associated therewith, and any other factors that may inform a decision to refer a particular medical provider.

At operation 1214, the process 1200 may include receiving from the computing device associated with the first medical provider a third indication of selection of a second medical provider of the one or more medical providers.

At operation 1216, the process 1200 may include processing the referral for the member to undergo the medical procedure based at least in part on at least one of the medical location or second medical provider. In some examples, processing the referral may include approving a cost associated with the procedure, scheduling the procedure, sending a message to the member informing the member and/or the first medical provider that the procedure is approved, sending a second message to the member and/or the computing device associated with the first medical provider and/or a second computing device associated with second medical provider to cause an appointment for the procedure to be scheduled.

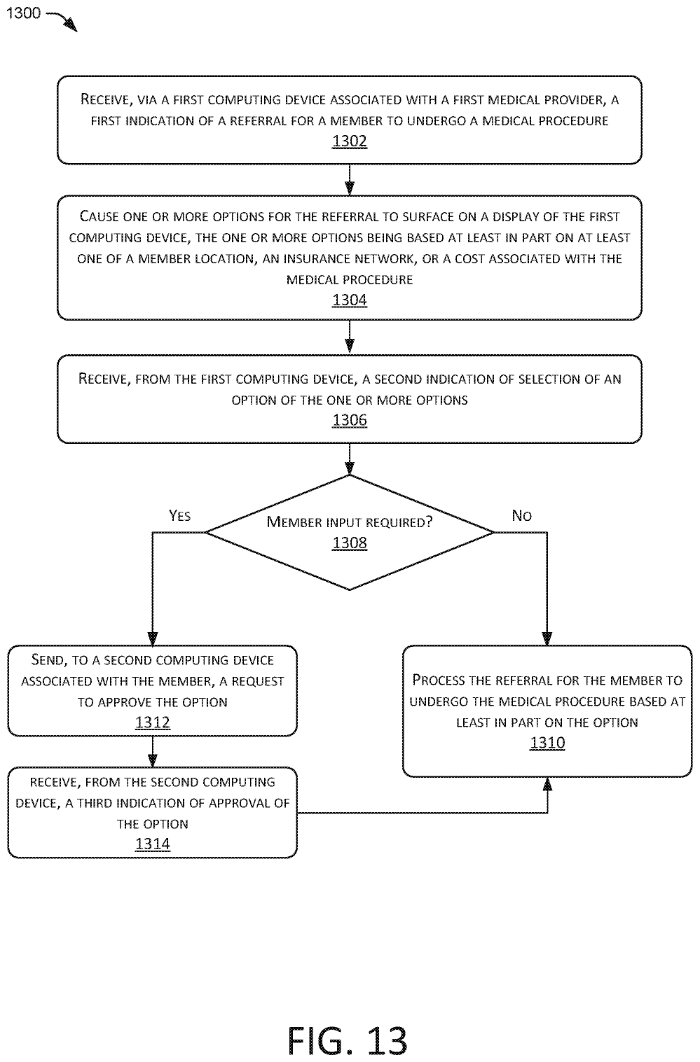

FIG. 13 illustrates an example process processing a referral based at least in part on input from a member. In some instances, some or all of process 1300 may be performed by one or more components in the systems 100 or 1000. By way of example and not limitation, the service provider computing device (e.g., service provider) referred to in process 1300 may be representative of a computing device associated with the service provider 104 or service provider server(s) 1002, the medical provider computing device (e.g., member device) referred to in process 1300 may be representative of the medical provider computing device(s) 106 and/or first computing device(s) 1004 and the member computing device referred to in process 1300 may be representative of the member computing device(s) 110 and/or the second computing device(s) 1006. However, the process 1300 is not limited to being performed by the system 100 or 1000.

At operation 1302, the process 1300 may include receiving, via a first computing device associated with a first medical provider, a first indication of a referral for a member to undergo a medical procedure. In some examples, the first indication of the referral may include an input provided via the window 612 of FIG. 6B, indicating that the medical provider would refer the member for the associated procedure (e.g., colonoscopy). In some examples, the first indication of the referral may include a selection of the selectable option 618 of FIG. 6B to submit a referral. In some examples, a first indication of the referral may include an indication that the medical provider has input member identifying information and/or a procedure via a website associated with a referral process, such as website 706 of FIG. 7.

At operation 1304, the process 1300 may include causing one or more options for the referral to surface on a display of the first computing device, the one or more options being based at least in part on at least one of a member location, an insurance network, or a cost associated with the medical procedure. In various examples, the options may include one or more locations and/or one or more providers associate with the procedure. The medical location(s) may include locations 624 and/or 722 of FIGS. 6C and 7, respectively. The medical provider(s) may include providers 626 and 724 of FIGS. 6C and 7, respectively.

As discussed above, the service provider may be configured to determine the member location based on a location associated with a member computing device, a member location associated with member data, and/or a location associated with the computing device associated with the first medical provider. In some examples, the medical location(s) and/or medical provider(s) may be determined based upon a location associated therewith being within a threshold distance and/or being one of the closest medical location(s) and/or medical provider(s) available to conduct the procedure.

In various examples, the medical location(s) and/or medical provider(s) may be identified based on a quality metric associated therewith. In some examples, the medical location(s) and/or medical provider(s) may be identified based on the respective quality metric being above a threshold quality metric.

At operation 1306, the process 1300 may include receiving, from the first computing device, a second indication of selection of an option of the one or more options. In some examples, the selection may include a selection associated with a medical location and/or medical provider. In some examples, the second indication of selection of the option may include a submission of the referral for the medical procedure.

At operation 1308, the process 1300 may include determining whether member input is required. In some examples, member input may be required based on a determination that a lower cost option is available. In such examples, the service provider may request approval from the member to accept the higher cost. In some examples the member input may be required based on a determination that the option includes a distance from a member location above a threshold distance (e.g., 50 miles, 100 miles, etc.). In such examples, the service provider may ensure the member approves of traveling the distance and/or that the current location of the member is different from a member location stored in the member data.

Based on a determination that the member input is not required (“No” at operation 1308), the process 1300, at operation 1310, may include processing the referral for the member to undergo the medical procedure based at least in part on the option.

Based on a determination that the member input is required (“Yes” at operation 1308), the process 1300, at operation 1312, may include sending, to a second computing device associated with the member, a request to approve the option. The request may include data associated with the referral, a cost associated with the option, costs associated with other options, and/or other information relevant to informing the member decision regarding the option.

At operation 1314, the process 1300 may include receiving from the second computing device, a third indication of approval of the option. Responsive to receiving third indication including approval of the option, the process 1300 may include, processing the referral for the member to undergo the medical procedure based at least in part on the option at operation 1310.

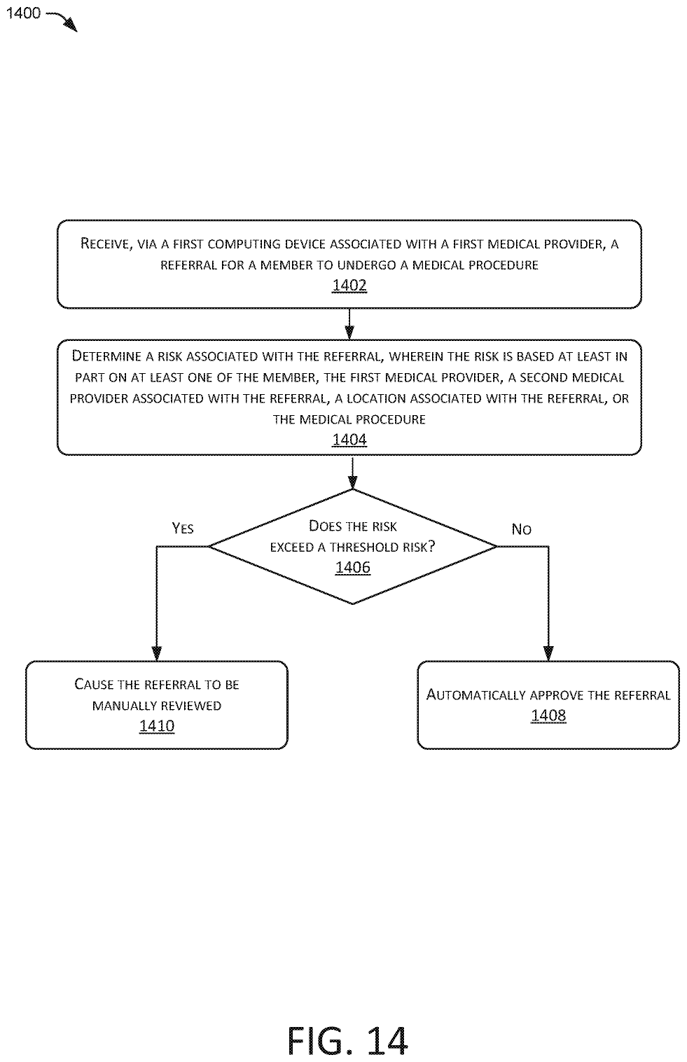

FIG. 14 illustrates an example processes 1400 for determining whether to automatically approve a referral, utilizing the techniques described herein. In some instances, some or all of process 1400 may be performed by one or more components in the systems 100 or 1000. By way of example and not limitation, the service provider computing device (e.g., service provider) referred to in process 1400 may be representative of a computing device associated with the service provider 104 or service provider server(s) 1002, the medical provider computing device (e.g., member device) referred to in process 1400 may be representative of the medical provider computing device(s) 106 and/or first computing device(s) 1004 and the member computing device referred to in process 1400 may be representative of the member computing device(s) 110 and/or the second computing device(s) 1006. However, the process 1400 is not limited to being performed by the system 100 or 1000.

At operation 1402, the process 1400 may include receiving, via a first computing device associated with a first medical provider, a referral for a member to undergo a medical procedure. In some examples, the referral may be submitted via an interface, such as interface 600C of FIG. 6C or interface 700 of FIG. 7.

At operation 1404, the process 1400 may include determining a risk associated with the referral, wherein the risk is based at least in part on at least one of the member, the first medical provider, a second medical provider associated with the referral, a location associate with the referral, or the medical procedure. In some examples, the risk may be based in part on member data, such as a payment history, health history, family health history, or the like. In some examples, the risk may be based on a quality metric associated with the first medical provider and/or the second medical provider. In some examples, the risk may be based on medical provider data associated with the first medical provider and/or the second medical provider, such as provider experience with a procedure, a number of procedures conducted per year, and the like. In various examples, the risk may be determined utilizing one or more machine learning models trained to output a risk associated with a referral, such as based on the factors described above. In such examples, the machine learning models may be trained utilizing training data associated with member data, medical provider data (e.g., location, experience, quality metric, etc.), and/or medical procedures.

At operation 1406, the process 1400 may include determining whether the risk exceeds a threshold risk.

Based on a determination that the risk does not exceed the threshold risk (“No” at operation 1406), the process 1400 may include, at operation 1408, automatically approving the referral. In some examples, the service provider may cause a notification to surface on a display of a medical provider device and/or a member device indicating the automatic approval.

Based on a determination that the risk does exceed the threshold risk (“Yes” at operation 1406), the process 1400 may include, at operation 1410, causing the referral to be manually reviewed. In various examples, an associate (e.g., employee, contractor, etc.) of the service provider may manually review the referral to determine whether to approve the referral.

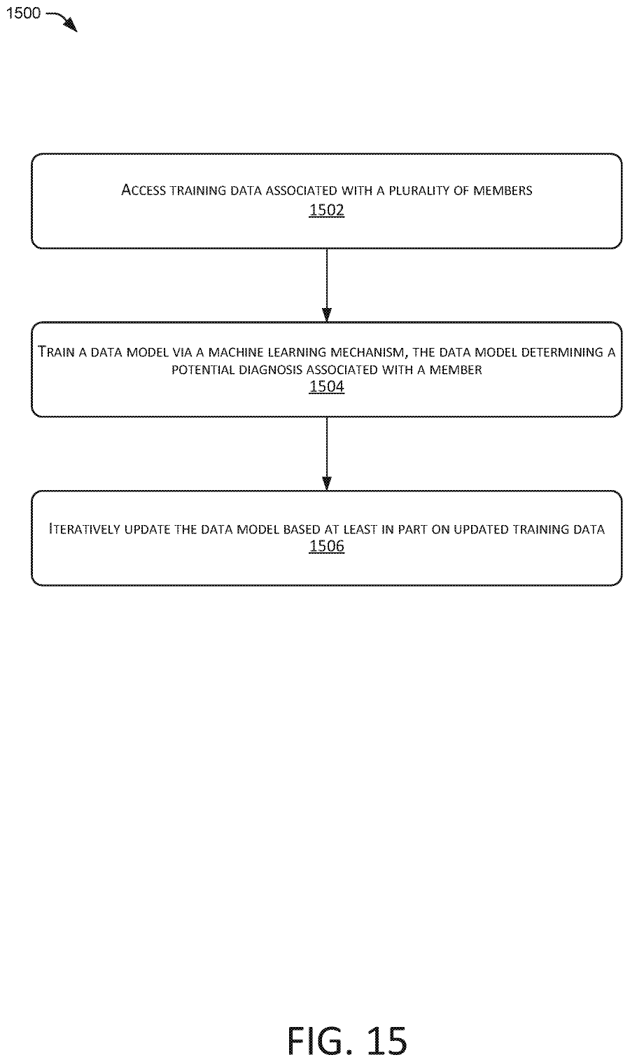

FIG. 15 illustrates an example processes 1500 for surfacing a clinical assessment tool and updating a member record based on input received via the clinical assessment tool, utilizing the techniques described herein. In some instances, some or all of process 1500 may be performed by one or more components in the systems 100 or 1000. By way of example and not limitation, the service provider computing device (e.g., service provider) referred to in process 1500 may be representative of a computing device associated with the service provider 104 or service provider server(s) 1002, the medical provider computing device (e.g., member device) referred to in process 1500 may be representative of the medical provider computing device(s) 106 and/or first computing device(s) 1004 and the member computing device referred to in process 1500 may be representative of the member computing device(s) 110 and/or the second computing device(s) 1006. However, the process 1500 is not limited to being performed by the system 100 or 1000.

At operation 1502, the process 1500 may include accessing training data associated with a plurality of members. The training data may include member data associated with the plurality of members.

At operation 1504, the process 1500 may include training a data model via a machine learning mechanism, the data model determining a potential diagnosis associated with a member. The data model may be trained utilizing supervised and/or unsupervised learning techniques. For example, machine learning techniques may include, but are not limited to, regression techniques (e.g., ordinary least squares regression (OLSR), linear regression, logistic regression, stepwise regression, multivariate adaptive regression splines (MARS), locally estimated scatterplot smoothing (LOESS)), instance-based techniques (e.g., ridge regression, least absolute shrinkage and selection operator (LASSO), elastic net, least-angle regression (LARS)), decisions tree techniques (e.g., classification and regression tree (CART), iterative dichotomiser 3 (ID3), Chi-squared automatic interaction detection (CHAID), decision stump, conditional decision trees), Bayesian techniques (e.g., naïve Bayes, Gaussian naïve Bayes, multinomial naïve Bayes, average one-dependence estimators (AODE), Bayesian belief network (BNN), Bayesian networks), clustering techniques (e.g., k-means, k-medians, expectation maximization (EM), hierarchical clustering), association rule learning techniques (e.g., perceptron, back-propagation, hopfield network, Radial Basis Function Network (RBFN)), deep learning techniques (e.g., Deep Boltzmann Machine (DBM), Deep Belief Networks (DBN), Convolutional Neural Network (CNN), Stacked Auto-Encoders), Dimensionality Reduction Techniques (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Techniques (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet50, ResNet101, VGG, DenseNet, PointNet, and the like.

At operation 1506, the process 1500 may include iteratively updating the data model based at least in part on updated training data.

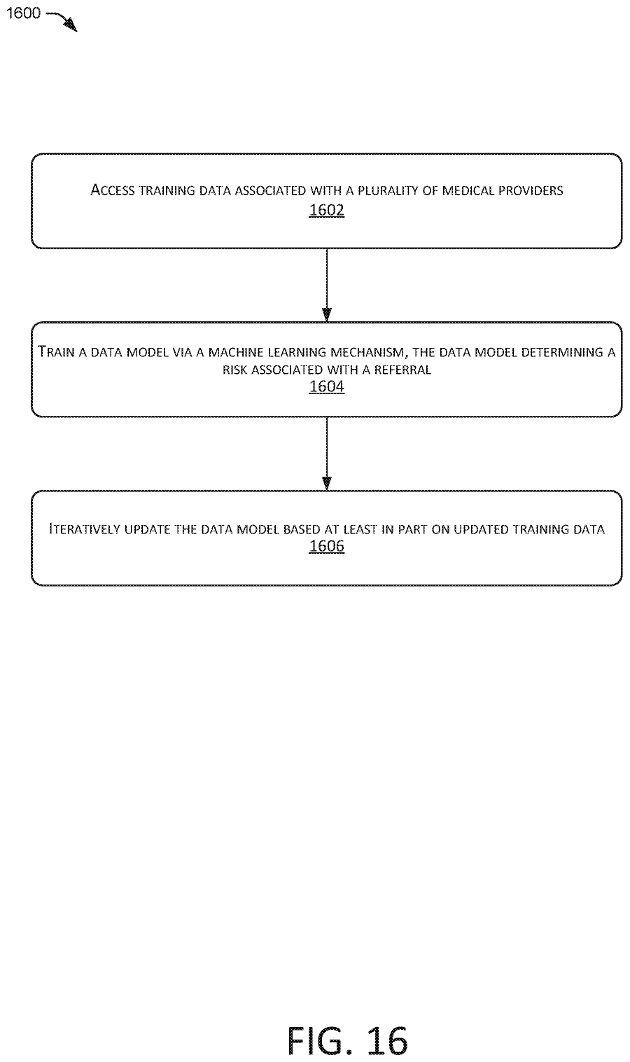

FIG. 16 illustrates an example processes 1600 for training a data model to determine whether a medical procedure may be automatically approved. In some instances, some or all of process 1600 may be performed by one or more components in the systems 100 or 1000. By way of example and not limitation, the service provider computing device (e.g., service provider) referred to in process 1600 may be representative of a computing device associated with the service provider 104 or service provider server(s) 1002, the medical provider computing device (e.g., member device) referred to in process 1600 may be representative of the medical provider computing device(s) 106 and/or first computing device(s) 1004 and the member computing device referred to in process 1600 may be representative of the member computing device(s) 110 and/or the second computing device(s) 1006. However, the process 1600 is not limited to being performed by the system 100 or 1000.

At operation 1602, the process 1600 may include accessing training data associated with a plurality of members. The training data may include member data associated with the plurality of members.

At operation 1604, the process 1600 may include training a data model via a machine learning mechanism, the data model determining a risk associated with a referral. The risk may be used to determine an automatic approval of the referral, such as that described above in FIG. 14. The data model may be trained utilizing supervised and/or unsupervised learning techniques.

At operation 1606, the process 1600 may include iteratively updating the data model based at least in part on updated training data.

As stated above, the order in which the operations are described is not intended to be construed as a limitation, and any number of the described operations may be combined in any order and/or in parallel to implement the processes. In some embodiments, one or more operations of the above-described methods may be omitted entirely. By way of example and not limitation, operations 1102 and 1104 may be performed without operations 1106 and 1108 and/or operations 1202-1208 and 1216 may be performed without operations 1212-1214. Moreover, the methods described herein can be combined in whole or in part with each other or with other methods.

The various techniques described herein may be implemented in the context of computer-executable instructions or software, such as program modules, that are stored in computer-readable storage and executed by the processor(s) of one or more computing devices such as those illustrated in the figures. Generally, program modules include routines, programs, objects, components, data structures, etc., and define operating logic for performing particular tasks or implement particular abstract data types.

Other architectures may be used to implement the described functionality and are intended to be within the scope of this disclosure. Furthermore, although specific distributions of responsibilities are defined above for purposes of discussion, the various functions and responsibilities might be distributed and divided in different ways, depending on circumstances.

Similarly, software may be stored and distributed in various ways and using different means, and the particular software storage and execution configurations described above may be varied in many different ways. Thus, software implementing the techniques described above may be distributed on various types of computer-readable media, not limited to the forms of memory that are specifically described.

### CONCLUSION

Although the discussion above sets forth example implementations of the described techniques, other architectures may be used to implement the described functionality, and are intended to be within the scope of this disclosure. Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A method comprising:
receiving, via a first instance of an application on a first computing device associated with a medical provider, a request to generate a clinical assessment associated with a clinical visit between the medical provider and a member;
generating the clinical assessment associated with the clinical visit, the clinical assessment including at least one of:
a potential diagnosis associated with the member;
a medication associated with the member;
a gap in care associated with the member; or
a clinical recommendation associated with the member;
causing the clinical assessment to surface via a second instance of the application on a second computing device associated with the medical provider;
receiving an indication that the clinical assessment is complete;
based at least in part on the indication that the clinical assessment is complete, causing a window to surface via at least one of the first instance of the application on the first computing device or the second instance of the application on the second computing, the window comprising at least one selectable option for indicating a means by which a primary medical record will be provided;
receiving the primary medical record; and
associating the primary medical record with the clinical assessment.

2. The method of claim 1, further comprising:
receiving an input corresponding to the at least one of the potential diagnosis, the medication, the gap in care, or the clinical recommendation; and
updating member data associated with the member based at least in part on the input.

3. The method of claim 1, wherein generating the clinical assessment comprises:
accessing member data associated with the member, the member data comprising at least one of a medical history, a laboratory result, a demographic, or a confirmed diagnosis;
determining the at least one of the potential diagnosis, the gap in care, or the clinical recommendation based at least in part on the member data.

4. The method of claim 1, wherein generating the clinical assessment comprises:
accessing medication information associated with the member, the medication information comprising at least one of a current medication prescribed to the member, an expired medication, or a fill history associated with medications prescribed to the member;
identify a potential modification to a medication;
wherein the clinical assessment includes the potential modification to the medication.

5. The method of claim 4, further comprising:
receiving, via the second instance of the application, an input corresponding to the potential modification; and
updating the medication information based at least in part on the input.

6. The method of claim 1, the further comprising:
determining at least one of:
a first time associated with the potential diagnosis;
a second time associated with the medication;
a third time associated with the gap in care;
a fourth time associated with the clinical recommendation; and
determining a total time associated with the clinical visit, the total time being based at least in part on the medical provider; and
determining whether to include the potential diagnosis, the medication, the gap in care, or the clinical recommendation in the clinical assessment based at least in part on the total time and the at least one of the first time, the second time, the third time, and the fourth time.

7. The method of claim 1, wherein generating the clinical assessment further comprises:
identifying at least one of:
first supporting evidence associated with the potential diagnosis,
second supporting evidence associated with the gap in care, or
third supporting evidence associated with the clinical recommendation; and
associating the at least one of the first supporting evidence with a first interface associated with the potential diagnosis, the second supporting evidence with a second interface associated with the gap in care, or the third supporting evidence with a third interface associated with the clinical recommendation
wherein causing the clinical assessment to surface via the second instance of the application comprises causing at least one of the first supporting evidence to surface via the first interface, the second supporting evidence to surface via the second interface, or the third supporting evidence to surface via the third interface.

8. The method of claim 1, further comprising determining at least one of the potential diagnosis, the gap in care or the clinical recommendation based at least in part on machine learning techniques.

9. A computing system comprising:
one or more processors; and
computer readable media storing instructions that, when executed by the one or more processors, cause the computing system to:
receive, via a first instance of an application on a first computing device associated with a medical provider, a first indication of a clinical visit between the medical provider and a member;
generate a clinical assessment associated with the clinical visit, the clinical assessment including at least one of:
a potential diagnosis associated with the member;
a medication associated with the member;
a gap in care associated with the member; or
a clinical recommendation associated with the member;
cause the clinical assessment to surface via a second instance of the application on a second computing device associated with the medical provide;
receive an indication that the clinical assessment is complete;
based at least in part on the indication that the clinical assessment is complete, cause a window to surface via at least one of the first instance of the application on the first computing device or the second instance of the application on the second computing, the window comprising at least one selectable option for indicating a means by which a primary medical record will be provided;
receive the primary medical record; and
associate the primary medical record with the clinical assessment.

10. The computing system of claim 9, wherein the clinical assessment includes a potential diagnosis, the instructions further causing the system to:
receive, via the second instance of the application, an indication of confirmation of the potential diagnosis;
update member data based at least in part on the indication of confirmation;
determine an option for treatment associated with the potential diagnosis; and
cause the option for treatment to surface via the second instance of the application on the second computing device associated with the medical provider.

11. The computing system of claim 9, wherein the clinical assessment includes a potential diagnosis, the instructions further causing the system to:
receive, via the second instance of the application, a first indication of inability to confirm the potential diagnosis;
determine one or more justifications associated with an inability to confirm the potential diagnosis;
receive a second indication associated with the inability to confirm the potential diagnosis; and
train the computing system to identify at least one of potential diagnoses or supporting evidence associated with the potential diagnoses based at least in part on the second indication.

12. The computing system of claim 9, wherein the clinical assessment includes a medication, the instructions further causing the system to:
determine a potential modification to the medication;
causing an indication of the potential modification to surface via the second instance of the application;
receiving, via the second instance of the application, an input corresponding to the potential modification; and
updating member data based at least in part on the input.

13. The computing system of claim 9, wherein the clinical assessment includes a gap in care, the instructions further causing the system to:
identify based at least in part on at least one of member data or clinical guidelines, that the member may be due for a procedure;
determine supporting evidence associated with the procedure, wherein the supporting evidence may include a last known date associated with the procedure, a clinical guideline associated with the procedure;
cause an indication of the procedure and the supporting evidence to surface on an interface associated with the second instance of the application.

14. The computing system of claim 9, wherein the clinical assessment includes a clinical recommendation, the instructions further causing the system to:
determine a current diagnosis associated with the member;
identify a treatment associated with the current diagnosis;
determine, based at least in part on member data associated with the member, that the treatment is not associated with a treatment plan corresponding to the member; and
cause the clinical assessment including the treatment to surface on an interface associated with the second instance of the application.

15. One or more computer readable media storing instructions that, when executed by one or more processors of a computing device, cause the computing device to:
receive, via a first instance of an application on a first computing device associated with a medical provider, a first indication of a clinical visit between the medical provider and a member;
generate a clinical assessment associated with the clinical visit, the clinical assessment including at least one of:
a potential diagnosis associated with the member;
a medication associated with the member;
a gap in care associated with the member; or
a clinical recommendation associated with the member;
cause the clinical assessment to surface via a second instance of the application on a second computing device associated with the medical provider;
receive an indication that the clinical assessment is complete;
based at least in part on the indication that the clinical assessment is complete, cause a window to surface via at least one of the first instance of the application on the first computing device or the second instance of the application on the second computing, the window comprising at least one selectable option for indicating a means by which a primary medical record will be provided;
receive the primary medical record; and
associate the primary medical record with the clinical assessment.

16. The one or more computer readable media of claim 15, wherein generating the clinical assessment further comprises:
identifying at least one of:
first supporting evidence associated with the potential diagnosis,
second supporting evidence associated with the gap in care, or
third supporting evidence associated with the clinical recommendation; and
associating the at least one of the first supporting evidence with a first interface associated with the potential diagnosis, the second supporting evidence with a second interface associated with the gap in care, or the third supporting evidence with a third interface associated with the clinical recommendation
wherein causing the clinical assessment to surface via the second instance of the application comprises causing at least one of the first supporting evidence to surface via the first interface, the second supporting evidence to surface via the second interface, or the third supporting evidence to surface via the third interface.

17. The one or more computer readable media of claim 15, the instructions further causing the computing device to:
determine at least one of:
a first time associated with the potential diagnosis;
a second time associated with the medication;
a third time associated with the gap in care;
a fourth time associated with the clinical recommendation; and
determine a total time associated with the clinical visit, the total time being based at least in part on the medical provider; and
determine whether to include the potential diagnosis, the medication, the gap in care, or the clinical recommendation in the clinical assessment based at least in part on the total time and the at least one of the first time, the second time, the third time, and the fourth time.

18. The one or more computer readable media of claim 15, the instructions further causing the computing device to:
receive an input corresponding to the at least one of the potential diagnosis, the medication, the gap in care, or the clinical recommendation; and
update member data associated with the member based at least in part on the input.

19. The one or more computer readable media of claim 15, the instructions further causing the computing device to:
access member data associated with the member, the member data comprising at least one of a medical history, a laboratory result, a demographic, or a confirmed diagnosis;
determine the at least one of the potential diagnosis, the gap in care, or the clinical recommendation based at least in part on the member data.

20. The one or more computer readable media of claim 15, wherein the clinical assessment includes a potential diagnosis, the instructions further causing the computing device to:
receive, via the second instance of the application, an indication of confirmation of the potential diagnosis;
update member data based at least in part on the indication of confirmation;
determine an option for treatment associated with the potential diagnosis; and
cause the option for treatment to surface via the second instance of the application on the second computing device associated with the medical provider.
